SEQUENCE LISTING (9 sequences)

SEQ ID NO: 1 - protein, 579 aa, Homo sapiens
MNAIVALCHFCELHGPRTLFCTEVLHAPLPQGDGNEDSPGQGEQAEEEEGGIQMNSRMRAHSPAEGASVESSSPGPKKSDMCEGCRSLAAGHPGYISHDKETSIKYVSHQHPSHPQLFSIVRQACVRSLSCEVCPGREGPIFFGDEQHGFVFSHTFFIKDSLARGFQRWYSIITIMMDRIYLINSWPFLLGKVRGIIDELQGKALKVFEAEQFGCPQRAQRMNTAFTPFLHQRNGNAARSLTSLTSDDNLWACLHTSFAWLLKACGSRLTEKLLEGAPTEDTLVQMEKLADLEEESESWDNSEAEEEEKAPVLPESTEGRELTQGPAESSSLSGCGSWQPRKLPVFKSLRHMRQVLGAPSFRMLAWHVLMGNQVIWKSRDVDLVQSAFEVLRTMLPVGCVRIIPYSSQYEEAYRCNFLGLSPHVQIPPHVLSSEFAVIVEVHAAARSTLHPVGCEDDQSLSKYEFVVTSGSPVAADRVGPTILNKIEAALTNQNLSVDVVDQCLVCLKEEWMNKVKVLFKFTKVDSRPKEDTQKLLSILGASEEDNVKLLKFWMTGLSKTYKSHLMSTVRSPTASESRN

SEQ ID NO: 2 - DNA, 20 bp, Artificial Sequence
  Description of Artificial Sequence Synthetic oligonucleotide
gaagtggcagagggcgacta

SEQ ID NO: 3 - DNA, 20 bp, Artificial Sequence
  Description of Artificial Sequence Synthetic oligonucleotide
tccgtgcagaagagcgtgcg

SEQ ID NO: 4 - DNA, 20 bp, Artificial Sequence
  Description of Artificial Sequence Synthetic oligonucleotide
atgtccgacttcttgggccc

SEQ ID NO: 5 - DNA, 3667 bp, Homo sapiens
ggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggatcctccagccccgtgagagggactgtcccttgagtttctcaactgctggaaggagctgtgtcccagcaaggaagggaaaccatcagggctgggctcggccctgtcaggtttggggcctgtgtgcttcccagactctccctccagccgttggaatcgctgaagatggcaatgaaaggcggagggatgatgggctctctctgtgttcaaactccttggagagacgactaggaggacagcttgcctcccaggccccttgtggacttagactcaaaacccgcaggagaaacaggtccgactcagtatgcagtcgcaataacatgtctgctcccgaggttaacattcaagcgtttctactttgaaattcagcaagagtttctgggccttatgtttgagggtaccttttgctgcagttgtgaatattcagtacattgccagctcttggtcactgagtgattgagttagggctccgcaagagactttggggagtgaagtggatctcttcctcatcttttggtcctctgaaatgtgtgttctgaagccatggggctcgtcttctggggtgttcccctgcaggtgctggtgaaggtaacctggggcttaatgatggagtccctgatcatttttgcacaagacaggttgctgaggggtcggcaagcatctgacttgcccaatcccctggatatggtgagccccgccatgcttttattctgtatcgcttttgtctttattgctgctttcaacatttacgtttggttacagttaactattttcggagtgtggtgattgaagacaatttcatcatcccactgtacttttttttttgagagggagtttcactcttgttgcccaggctggagtgcaatggcacgatcttggctcactgcaacctctgcctcctgggttcaagcaattctcctgcctcagcctccagagtagctggaactacaggtgcccgccactatgcccagctaatttttgtattttttagtagagacggggtttcaccgtgttggccgggctggtctcaaactcctgacctcaggtgatccacccacctcagcctcccaaagtgctgggattacaagcgtgagccactgtgcctggcccttttttttttttttttttttttttttaaagagatggcatcttgctatgtcgtccaggctggtcttgaactcctgagttcaagcagtcctcctgcttcaacatacagctacaggtaccccccactatacatttttaataaggattcatggctcagagggattttctgatggttttgctgatttgtttctagtttttttgtgtttatatttaacatgaagaccaagtttatataactaggtatctgtataatgcaacaacattggaacacaataaagatgtatttttgtaaa

SEQ ID NO: 6 - DNA, 3325 bp, Homo sapiens
ggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctggtgaggcaggggtgctgttgccggggccttggcccggatggccgtggggcggtaccagctgtctgctctcctggcaggaatcgctgagggagggaaacgcggctctgaatcagcccagaacgagccttcgggaagctcaccctccgatctcggtgtgattgttgtgattgttgtgatttcctgtctcgtttgccttgaccgccatgtgaaagaatctgttccccagctaggtggggaaaattcacaggtgggctgtctgtagagagaactggctgattaaaggcttctcgtcccgattttgtgatagccaagtgcttggcctggtcgacggtctttgctcctttacaaataaagtgttctgtttcagttcgtcccaagttttccatgaagggcagtggttccctgacctcccaggtgcctgggcttccccaggttcctgatctggggcttggggccctgtgtttggggatcgtggcactgtgtgcaccagcctggaagcactgggccagtcttggccaagctttccatcagggatgatttgatcttggtgctacaggtctgtggtacgaccattgttccacaccacatgtcattaataatgcttcccatgcttctgcttgcaaatgaccagccttccaaacagccagagctgtttcgaggtgtttctgcaggcaggtgcaggcgtgccctcaaataagctttgccaatggagtctcagcaagagcaaaacctggtcaggaaagacaaagcctgggaatccacccccatgccctgcaggttggctggccctggagccatttattatagtgctaatcatgtttctaggcaggtgcagatggcaagggcagtgtcttggtgagctttttagcacgaagagccaggtctgtcgaagcctttgtgagagctggaaacgcaggtgtgctgggcatgcgcagtatggggtttcgggctcagggcttgccctttggcatcagacagacctggcttcgcatcctggatttgcttctgacgtgcacccttccctttgggtctcgtgatgtgaaatggagatgttgtcatttgtgagggctccatgaagtttcgttgaaatgacaaatactaatttcttcatctgtgaaatggagataatagtgctgacctcagaacagctgagaggactaaatgaaatgatgttggatgtagccataaagaacgaagtcaggcactggtgcacgcctggaatcccagctcttgggagaccgagacaggtggattgcttgagctcaggagtttgagaccagcctgagcaacatagggaggtccagtctctacaaaaaatatgaaaagtagctgggcgtggtggcgcatgcctgtagtcccactacttggaaggcttcgttgggaggatcacttgagcccagaagattgaggctgcagtaagccgtgatcgtgccactgcattccagcctgggcaacagagcgagacactgtctcaaataaaaaagatgggaatagtagacactgggggctccagaaggagggagggagggaggaaggggaggaagggctgaaatgctttctattggatactatctgggcatattacttcctgtggttcactgtctgggtgacaggattcatagaagcccaaactttagcaccacgcagcatacccttgtaacaaagccgcacacgtacgccctcaagctaaaacaaaagtggaccgggaggccgaggtcgggggatcatgaggtcaggagtttgagaccagcctggcagataacggtgaaaccccgtctctactaaaaataccaaaaaaagttagccggacatggtggcaggtgcctgtagtcccagctacttgggaggctggggcagaagaatcgcttgaacccaggaggcggaggttgcagtgagccgagattgcgccactgcactccagcctgtgcgacagagtgagactccgtctcaaaaaaaaaaaaaaaaaaaaa

SEQ ID NO: 7 - DNA, 3929 bp, Homo sapiens
ggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcaggttttgtcttcgctctgtttggaggagagggtgtgtgtcatcctcttctcccagtttggcgttcaggagggtcctctgatgcgctaatagggtagcaccgtgtcctccagggagggtggaagaccgcgcttctctccagttgagagtactgtcagtcgcgtccttgtctcctggaaagaatggattggcttgtggattgaagtccaagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgagtgcagggagagtgcagtggctgctctgacctgctggttcttctgcatgctccaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggatcctccagccccgtgagagggactgtcccttgagtttctcaactgctggaaggagctgtgtcccagcaaggaagggaaaccatcagggctgggctcggccctgtcaggtttggggcctgtgtgcttcccagactctccctccagccgttggaatcgctgaagatggcaatgaaaggcggagggatgatgggctctctctgtgttcaaactccttggagagacgactaggaggacagcttgcctcccaggccccttgtggacttagactcaaaacccgcaggagaaacaggtccgactcagtatgcagtcgcaataacatgtctgctcccgaggttaacattcaagcgtttctactttgaaattcagcaagagtttctgggccttatgtttgagggtaccttttgctgcagttgtgaatattcagtacattgccagctcttggtcactgagtgattgagttagggctccgcaagagactttggggagtgaagtggatctcttcctcatcttttggtcctctgaaatgtgtgttctgaagccatggggctcgtcttctggggtgttcccctgcaggtgctggtgaaggtaacctggggcttaatgatggagtccctgatcatttttgcacaagacaggttgctgaggggtcggcaagcatctgacttgcccaatcccctggatatggtgagccccgccatgcttttattctgtatcgcttttgtctttattgctgctttcaacatttacgtttggttacagttaactattttcggagtgtggtgattgaagacaatttcatcatcccactgtacttttttttttgagagggagtttcactcttgttgcccaggctggagtgcaatggcacgatcttggctcactgcaacctctgcctcctgggttcaagcaattctcctgcctcagcctccagagtagctggaactacaggtgcccgccactatgcccagctaatttttgtattttttagtagagacggggtttcaccgtgttggccgggctggtctcaaactcctgacctcaggtgatccacccacctcagcctcccaaagtgctgggattacaagcgtgagccactgtgcctggcccttttttttttttttttttttttttttaaagagatggcatcttgctatgtcgtccaggctggtcttgaactcctgagttcaagcagtcctcctgcttcaacatacagctacaggtaccccccactatacatttttaataaggattcatggctcagagggattttctgatggttttgctgatttgtttctagtttttttgtgtttatatttaacatgaagaccaagtttatataactaggtatctgtataatgcaacaacattggaacacaataaagatgtatttttgtaaa

SEQ ID NO: 8 - DNA, 3950 bp, Homo sapiens
ggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcagaacccccaaagcctccccagtctctgataacacatccagggtctttcggtggcctggaggagctataagatctgcttcctacagcctccaccctgtctgctgctgctctttggacaccggaccttacacacccctcagacagtacagcacccaccctgcccttcaactcttgcttggccccgtaagccagtccttccaggctcctgcctcctctggtcattccctgcgtgggcctctcgcaggaagcagttttcctgtgtcccccgccgaccccctgcaggaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggatcctccagccccgtgagagggactgtcccttgagtttctcaactgctggaaggagctgtgtcccagcaaggaagggaaaccatcagggctgggctcggccctgtcaggtttggggcctgtgtgcttcccagactctccctccagccgttggaatcgctgaagatggcaatgaaaggcggagggatgatgggctctctctgtgttcaaactccttggagagacgactaggaggacagcttgcctcccaggccccttgtggacttagactcaaaacccgcaggagaaacaggtccgactcagtatgcagtcgcaataacatgtctgctcccgaggttaacattcaagcgtttctactttgaaattcagcaagagtttctgggccttatgtttgagggtaccttttgctgcagttgtgaatattcagtacattgccagctcttggtcactgagtgattgagttagggctccgcaagagactttggggagtgaagtggatctcttcctcatcttttggtcctctgaaatgtgtgttctgaagccatggggctcgtcttctggggtgttcccctgcaggtgctggtgaaggtaacctggggcttaatgatggagtccctgatcatttttgcacaagacaggttgctgaggggtcggcaagcatctgacttgcccaatcccctggatatggtgagccccgccatgcttttattctgtatcgcttttgtctttattgctgctttcaacatttacgtttggttacagttaactattttcggagtgtggtgattgaagacaatttcatcatcccactgtacttttttttttgagagggagtttcactcttgttgcccaggctggagtgcaatggcacgatcttggctcactgcaacctctgcctcctgggttcaagcaattctcctgcctcagcctccagagtagctggaactacaggtgcccgccactatgcccagctaatttttgtattttttagtagagacggggtttcaccgtgttggccgggctggtctcaaactcctgacctcaggtgatccacccacctcagcctcccaaagtgctgggattacaagcgtgagccactgtgcctggcccttttttttttttttttttttttttttaaagagatggcatcttgctatgtcgtccaggctggtcttgaactcctgagttcaagcagtcctcctgcttcaacatacagctacaggtaccccccactatacatttttaataaggattcatggctcagagggattttctgatggttttgctgatttgtttctagtttttttgtgtttatatttaacatgaagaccaagtttatataactaggtatctgtataatgcaacaacattggaacacaataaagatgtatttttgtaaa

SEQ ID NO: 9 - DNA, 3866 bp, Homo sapiens
ggcggggctgcgggaccgcgagtgagtgtggtcgctcctggttctgccagctcccctgagagcctgaacccgggcttgagagcctcgccaccccgggtgacatccctgccgtgggcttgggggctctgggtgtgattccgccggtccgggtcccgcagcgaccacctacccagcgcagtcaggggcggggctgggacccagagcgggaccccggctgccgagtccaggtgtcccgcgggcctcgatttggggagcagaaaacgccaggtcttcaagggtgtctgccaccaccatgcctgacccatttggcagcagcctcgtgtgtggtggtctggtgtggacggtggaagcgtgattctgctgagtgtcagtgtgaccactcgtgctcagccgtatctcagcaggaggacaggtgccggagcagctcgtgcagctaagcagccaactgcagaaacgtcagcctccaccctgtctgctgctgctctttggacaccggaccttacacacccctcagacagtacagcacccaccctgcccttcaactcttgcttggccccgtaagccagtccttccaggctcctgcctcctctggtcattccctgcgtgggcctctcgcaggaagcagttttcctgtgtcccccgccgaccccctgcaggaggcctgttgcagtctccaaggcaccatgaatgccatcgtggctctctgccacttctgcgagctccacggcccccgcactctcttctgcacggaggtgctgcacgccccacttcctcaaggggatgggaatgaggacagtcctggccagggtgagcaggcggaagaagaggaaggtggcattcagatgaacagtcggatgcgtgcgcacagccccgcagagggggccagcgtcgagtccagcagcccggggcccaaaaagtcggacatgtgcgagggctgccggtcacttgctgcagggcacccgggatatatcagccatgataaagagacctccattaaatacgtcagccaccagcaccccagccacccccagctcttcagcattgtccgccaggcctgtgtccggagcctgagctgtgaggtctgccctggccgtgaaggccccatcttcttcggagatgagcagcacggctttgtgttcagccacaccttcttcatcaaggacagcctggccaggggcttccagcgctggtacagcatcatcaccatcatgatggaccggatctacctcatcaactcctggcccttcctgctggggaaggtccggggaatcatcgatgagctccagggcaaggcgctcaaggtgtttgaggcagagcagtttggatgcccacagcgtgctcagaggatgaacacagccttcacgccattcctacaccagaggaacggcaacgccgcccgctcgctgacatcgctgacaagtgatgacaacctgtgggcgtgcctgcacacctcctttgcctggctcctgaaggcgtgtggcagccggctgaccgagaagctcctggaaggtgctccgaccgaggataccttggtccagatggagaagctcgctgatttagaagaggaatcagaaagctgggacaactctgaggctgaagaggaggagaaagcccctgtgttgccagagagtacagaagggcgggagctgacccagggcccggcagagtcctcctctctctcaggctgtgggagctggcagccccggaagctgccagtcttcaagtccctccggcacatgaggcaggtcctgggtgccccttctttccgcatgctggcctggcacgttctcatggggaaccaggtgatctggaaaagcagagacgtggacctcgtccagtcagcttttgaagtacttcggaccatgcttcccgtgggctgcgtccgcatcatcccatacagcagccagtacgaggaggcctatcggtgcaacttcctggggctcagcccgcacgtgcagatccccccccacgtgctctcctcagagtttgctgtcatcgtggaggtccacgcagccgcacgttccaccctccaccctgtggggtgtgaggatgaccagtctctcagcaagtacgagtttgtggtgaccagtgggagccctgtagctgcagaccgagtgggccccaccatcctgaataagattgaagcggctctgaccaaccagaacctgtctgtggatgtggtggaccagtgcctcgtctgcctcaaggaggagtggatgaacaaagtgaaggtgctttttaagttcaccaaggtggacagtcgacccaaagaggacacacagaagctgctgagcatcctgggtgcgtccgaggaggacaatgtcaagctgctgaagttctggatgactggcctgagcaagacctacaagtcacacctcatgtccacggtccgcagccccacagcctcggagtctcggaactgacccgtcacacacacctgcctaaagacagggatggctgtccacaggatcctccagccccgtgagagggactgtcccttgagtttctcaactgctggaaggagctgtgtcccagcaaggaagggaaaccatcagggctgggctcggccctgtcaggtttggggcctgtgtgcttcccagactctccctccagccgttggaatcgctgaagatggcaatgaaaggcggagggatgatgggctctctctgtgttcaaactccttggagagacgactaggaggacagcttgcctcccaggccccttgtggacttagactcaaaacccgcaggagaaacaggtccgactcagtatgcagtcgcaataacatgtctgctcccgaggttaacattcaagcgtttctactttgaaattcagcaagagtttctgggccttatgtttgagggtaccttttgctgcagttgtgaatattcagtacattgccagctcttggtcactgagtgattgagttagggctccgcaagagactttggggagtgaagtggatctcttcctcatcttttggtcctctgaaatgtgtgttctgaagccatggggctcgtcttctggggtgttcccctgcaggtgctggtgaaggtaacctggggcttaatgatggagtccctgatcatttttgcacaagacaggttgctgaggggtcggcaagcatctgacttgcccaatcccctggatatggtgagccccgccatgcttttattctgtatcgcttttgtctttattgctgctttcaacatttacgtttggttacagttaactattttcggagtgtggtgattgaagacaatttcatcatcccactgtacttttttttttgagagggagtttcactcttgttgcccaggctggagtgcaatggcacgatcttggctcactgcaacctctgcctcctgggttcaagcaattctcctgcctcagcctccagagtagctggaactacaggtgcccgccactatgcccagctaatttttgtattttttagtagagacggggtttcaccgtgttggccgggctggtctcaaactcctgacctcaggtgatccacccacctcagcctcccaaagtgctgggattacaagcgtgagccactgtgcctggcccttttttttttttttttttttttttttaaagagatggcatcttgctatgtcgtccaggctggtcttgaactcctgagttcaagcagtcctcctgcttcaacatacagctacaggtaccccccactatacatttttaataaggattcatggctcagagggattttctgatggttttgctgatttgtttctagtttttttgtgtttatatttaacatgaagaccaagtttatataactaggtatctgtataatgcaacaacattggaacacaataaagatgtatttttgtaaa

(19) United States
(12) Patent Application Publication
(10) Pub. No.: US 2021/0301287 A1
(43) Pub. Date: Sep. 30, 2021

(54) Treatment Of Non-Alcoholic Fatty Liver Disease, Non-Alcoholic Steatohepatitis, And Liver Fibrosis

(71) Applicant: The Trustees of the University of Pennsylvania, Philadelphia, PA (US)

(72) Inventors: Zoltan Arany, Merion Station, PA (US); Bridget Gosis, Philadelphia, PA (US)

(21) Appl. No.: 17/156,348

(22) Filed: Jan. 22, 2021

Related U.S. Application Data
(60) Provisional application No. 62/964,534, filed on Jan. 22, 2020.

(51) Int. Cl. C12N 15/11; C12N 9/22; C12N 15/113

(52) U.S. Cl. CPC C12N 15/111; C12N 9/22; C12N 15/1135; C12N 2310/3535; C12N 2320/32; C12N 2310/14; C12N 2310/20

(57) ABSTRACT

Provided herein are compositions for preventing or treating nonalcoholic fatty liver disease or nonalcoholic steatohepatitis in a subject comprising an inhibitor of folliculin, methods for preventing or treating nonalcoholic fatty liver disease or nonalcoholic steatohepatitis in a subject comprising administering to the liver of the subject an inhibitor of folliculin, and methods for preventing or treating nonalcoholic fatty liver disease or nonalcoholic steatohepatitis in a subject comprising silencing or down-regulating FLCN in hepatocytes of the subject.

### CROSS-REFERENCE TO RELATED APPLICATIONS

This application claims the benefit of U.S. Provisional Application No. 62/964,534, filed Jan. 22, 2020, the entire contents of which is incorporated herein by reference for any and all purposes.

### GOVERNMENT RIGHTS

This invention was made with government support under Contract No. F30 DK120096 awarded by the National Institutes of Health—National Institute of Diabetes and Digestive and Kidney Diseases. The government has certain rights in the invention.

### TECHNICAL FIELD

The present disclosure relates to prevention and treatment of liver disease using an inhibitor of folliculin.

### BACKGROUND

Nonalcoholic fatty liver disease (NAFLD) represents a major public health issue affecting approximately 30% of individuals in the U.S. If left untreated, NAFLD can progress to nonalcoholic steatohepatitis (NASH), and consequently to cirrhosis and hepatocellular carcinoma (HCC). NAFLD is a complex disease involving genetic and environmental factors. While there have been many studies proposing divergent molecular bases for NAFLD, the mechanism is still largely not understood. Currently, no specific pharmacologic therapy exists for NAFLD or NASH. On this spectrum of disease, the FDA has in particular highlighted the need to target NASH, as it best predicts long-term adverse outcomes like liver failure and HCC.

Accordingly, there exists an urgent outstanding need for therapies that can prevent or treat nonalcoholic fatty liver disease and nonalcoholic steatohepatitis.

### SUMMARY

Provided herein are compositions for preventing or treating nonalcoholic fatty liver disease or nonalcoholic steatohepatitis in a subject comprising an inhibitor of folliculin. The inhibitor of folliculin may be an siRNA or a Cas9 enzyme and a guide RNA (gRNA) that specifically binds to, recognizes, or hybridizes to FLCN.

Also provided are methods for preventing or treating nonalcoholic fatty liver disease or nonalcoholic steatohepatitis in a subject comprising administering to the liver of the subject an inhibitor of folliculin, such as e.g. an siRNA or a Cas9 enzyme and a guide RNA (gRNA) that specifically binds to, recognizes, or hybridizes to FLCN.

The present disclosure also pertains to methods for preventing or treating nonalcoholic fatty liver disease or nonalcoholic steatohepatitis in a subject comprising silencing or down-regulating FLCN in hepatocytes of the subject. The FLCN may be downregulated using an inhibitor of folliculin. The inhibitor of folliculin may be an siRNA or a Cas9 enzyme and a guide RNA (gRNA) that specifically binds to, recognizes, or hybridizes to FLCN.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

The summary, as well as the following detailed description, is further understood when read in conjunction with the appended drawings. For the purpose of illustrating the disclosed compositions, methods, and kits there are shown in the drawings exemplary embodiments of compositions, methods, and kits; however, these should not be limited to the specific embodiments disclosed. In the drawings:

FIGS. 1A-1H provide the results of a study regarding the results of deleting the FLCN gene for protection against nonalcoholic fatty liver disease (NAFLD).

FIGS. 2A-2F illustrate the results of a study demonstrating that FLCN deletion protects against and reverses non-alcoholic steatohepatitis (NASH).

FIGS. 3A-3D pertain to the results of a study demonstrating that FLCN coordinates several hepatic lipid processes, and FLCN deletion unleashes TFE3 to protect against NAFLD via multiple mechanisms.

FIGS. 4A-4D provide the results of a study demonstrating that FLCN KO mice are protected from steatosis via TFE3 activation.

FIG. 5 shows a Western Blot of AML12 cells infected with lentivirus containing various Flcn guide RNAs (cloned into the lentiCRISPR v2 puro backbone).

### DETAILED DESCRIPTION OF ILLUSTRATIVE EMBODIMENTS

The present disclosure is based on the discovery that through inhibition of folliculin it is possible to prevent or treat nonalcoholic fatty liver disease and/or nonalcoholic steatohepatitis in a subject. For instance, the disclosure is based on the discovery that through down-regulating and/or silencing FLCN using an siRNA or a guide RNA it is possible prevent or treat nonalcoholic fatty liver disease and/or nonalcoholic steatohepatitis in a subject.

The present inventions may be understood more readily by reference to the following detailed description taken in connection with the accompanying examples, which form a part of this disclosure. It is to be understood that these inventions are not limited to the specific formulations, methods, articles, or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

### Definitions

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a component” is a reference to one or more of such reagents and equivalents thereof known to those skilled in the art, and so forth. Furthermore, when indicating that a certain element “may be” X, Y, or Z, it is not intended by such usage to exclude in all instances other choices for the element.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” can refer to a value of 7.2 to 8.8, inclusive. This value may include “exactly 8”. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

An “inhibitor” of a protein, e.g., of folliculin, may refer to a moiety that down regulates or silences the gene that produces the protein (e.g., the FLCN gene), or may refer to a moiety that interferes with the ability of the protein to function in its prescribed (wild-type) manner. In certain instances, the inhibitor only down regulates or silences the gene that produces the protein and does not affect the protein itself.

As used herein, the terms “treatment” or “therapy” (as well as different word forms thereof) includes preventative (e.g., prophylactic), curative, or palliative treatment. Such preventative, curative, or palliative treatment may be full or partial. For example, complete elimination of unwanted symptoms, or partial elimination of one or more unwanted symptoms would represent “treatment” as contemplated herein.

As employed throughout the present disclosure, the term “effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with the particular compound, component or composition selected, the route of administration, and the ability of the components to elicit a desired response in the individual, but also with factors such as the disease state or severity of the condition to be alleviated, hormone levels, age, sex, weight of the individual, the state of being of the patient, and the severity of the condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the components are outweighed by the therapeutically beneficial effects. As an example, the compositions useful in the methods of the present invention are administered at a dosage and for a time such that the level of the production of folliculin is reduced as compared to the level before the start of treatment.

As used herein “silencing or down-regulating FLCN” includes silencing or down-regulating FLCN gene expression.

The term “RNA” as used herein is defined as ribonucleic acid.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

Nucleic acids can be single stranded or double-stranded or can contain portions of both double-stranded and single-stranded sequence. The nucleic acid can be DNA, both genomic and cDNA, RNA, or a hybrid, where the nucleic acid can contain combinations of deoxyribo and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

“Complement” or “complementary” as used herein means Watson-Crick (e.g., A-T/U and CG) or Hoogsteen base pairing between nucleotides or nucleotide analogs of nucleic acid molecules.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. Identity can be performed manually or by using a computer sequence algorithm such as BLAST or BLAST 2.0.

“Vector” as used herein means a nucleic acid sequence containing an origin of replication. A vector can be a viral vector, bacteriophage, bacterial artificial chromosome, or yeast artificial chromosome. A vector can be a DNA or RNA vector. A vector can be a self-replicating extrachromosomal vector, and preferably, is a DNA plasmid.

The term “CRISPR/CAS,” “clustered regularly interspaced short palindromic repeats system,” or “CRISPR” refers to DNA loci containing short repetitions of base sequences. Each repetition is followed by short segments of spacer DNA from previous exposures to a virus. Bacteria and archaea have evolved adaptive immune defenses termed CRISPR-CRISPR-associated (Cas) systems that use short RNA to direct degradation of foreign nucleic acids. In bacteria, the CRISPR system provides acquired immunity against invading foreign DNA via RNA-guided DNA cleavage.

In the type II CRISPR/Cas system, short segments of foreign DNA, termed “spacers” are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Recent work has shown that target recognition by the Cas9 protein requires a “seed” sequence within the crRNA and a conserved dinucleotide-containing protospacer adjacent motif (PAM) sequence upstream of the crRNA-binding region.

To direct Cas9 to cleave sequences of interest, crRNA-tracrRNA fusion transcripts, hereafter referred to as “guide RNAs” or “gRNAs” may be designed, from human U6 polymerase III promoter. CRISPR/CAS mediated genome editing and regulation, highlighted its transformative potential for basic science, cellular engineering, and therapeutics.

The term “CRISPRi” refers to a CRISPR system for sequence specific gene repression or inhibition of gene expression, such as at the transcriptional level.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

### Description

The present disclosure includes compositions, methods, and kits for preventing or treating preventing or treating nonalcoholic fatty liver disease or nonalcoholic steatohepatitis in a subject using an inhibitor of folliculin. The siRNA or a Cas9 enzyme and a guide RNA (gRNA) that specifically binds to, recognizes, or hybridizes to FLCN.

Provided herein are compositions for preventing or treating nonalcoholic fatty liver disease or nonalcoholic steatohepatitis in a subject comprising an inhibitor of folliculin. In one embodiment, the compositions are for preventing or treating nonalcoholic fatty liver disease or nonalcoholic steatohepatitis. In another embodiment, the compositions are for treating nonalcoholic fatty liver disease or nonalcoholic steatohepatitis.

Also disclosed are methods for preventing or treating nonalcoholic fatty liver disease or nonalcoholic steatohepatitis in a subject comprising administering to the liver of the subject an inhibitor of folliculin. Accordingly, one embodiment of the invention is directed to a method of preventing nonalcoholic fatty liver disease and/or nonalcoholic steatohepatitis in a subject comprising administering to the liver of the subject an inhibitor of folliculin. Another embodiment of the invention is directed to a method treating nonalcoholic fatty liver disease and/or nonalcoholic steatohepatitis in a subject comprising administering to the liver of the subject an inhibitor of folliculin.

Also provided herein are methods for preventing or treating nonalcoholic fatty liver disease or nonalcoholic steatohepatitis in a subject comprising silencing or down-regulating FLCN in hepatocytes of the subject. Accordingly, one embodiment of the invention is a method of preventing nonalcoholic fatty liver disease or nonalcoholic steatohepatitis in a subject comprising silencing or down-regulating FLCN in hepatocytes of the subject. In one embodiment, the method silences the FLCN gene in hepatocytes of the subject. In another embodiment, the method down-regulates FLCN, e.g. the FLCN gene, in hepatocytes of the subject. Another embodiment of the invention is a method of treating nonalcoholic fatty liver disease or nonalcoholic steatohepatitis in a subject comprising silencing or down-regulating FLCN, e.g. the FLCN gene, in hepatocytes of the subject. In one embodiment, the method silences the FLCN, e.g. the FLCN gene, in hepatocytes of the subject. In another embodiment, the method down-regulates the FLCN, e.g. the FLCN gene, in hepatocytes of the subject.

The present disclosure pertains to the discovery that liver-specific FLCN deletion powerfully protects against diet-induced steatosis in mice; surprisingly, this observation held under diets that promote steatosis through different mechanisms. When challenged with diets that activate de novo lipogenesis (DNL), FLCN-null mice highly suppress DNL. However, under diet conditions that block secretion of very-low density lipoprotein-triacylglycerol (VLDL-TAG) particles, FLCN-null mice displayed unchanged DNL yet upregulated VLDL-TAG secretion. These data indicate that mice lacking hepatic FLCN can adapt to distinct drivers of NAFLD through altering different pathways of lipid homeostasis. Mechanistically, it was found that FLCN-null mice avert steatosis through activation of TFE3. Investigations indicated that TFE3 transcriptionally activates VLDL-TAG secretion and fatty acid oxidation genes, while also suppressing DNL genes, most likely through inhibition of the transcription factor SREBP-1c. Finally, it was observed that FLCN liver deletion also protects against and reverses NASH, a critical intermediate in the liver disease spectrum. In summary, these studies reveal a powerful role for FLCN in liver lipid metabolism.

Pursuant to the present compositions and methods, the inhibitor of folliculin may be targeted to hepatocytes of the subject. In order to target hepatocytes, the inhibitor of folliculin may be a GalNac-modified oligonucleotide. In some embodiments, the inhibitor of folliculin operates by targeting the FLCN gene.

The inhibitor of folliculin may operate by RNA interference (RNAi). The inhibitor of folliculin may also operate via CRISPR.

RNAi is widely used by researchers to silence genes to learn something about their function. In certain embodiments, the inhibitor of folliculin is small interfering RNA (siRNA). siRNAs can be designed to match any gene, can be manufactured cheaply, and can be readily administered to cells. In fact, it is now possible to order commercially synthesized siRNAs to silence virtually any gene in a human or other organism's cell, dramatically accelerating the pace of biomedical research. The siRNA may an a double-stranded RNA molecule having a sequence homologous with the nucleotide sequence of mRNA which is transcribed from the FLCN gene, and a sequence complementary with the FLCN nucleotide sequence. The siRNA generally is homologous/complementary with one region of mRNA which is transcribed from the gene or may be siRNA including a plurality of RNA molecules which are homologous/complementary with different regions.

In the present compositions and methods, siRNA can be used to silence the FLCN gene. The siRNA is complementary to a portion of the nucleic acid sequence of FLCN and downregulates and/or inhibits expression of FLCN. In certain embodiments, the siRNA is complementary to a portion of the nucleic acid sequence of FLCN transcript variant 1-5 (SEQ ID NO: 5-9) and downregulates and/or inhibits expression of FLCN. In another embodiment, the siRNA is complementary to a portion of the nucleic acid encoding the protein of SEQ ID NO: 1, and downregulates and/or inhibits expression of FLCN.

```
Homo Sapiens folliculin
(SEQ ID NO: 1)
    1 mnaivalchf celhgprtlf ctevlhaplp qgdgnedspg qgeqaeeeeg giqmnsrmra
  61 hspaegasve ssspgpkksd mcegcrslaa ghpgyishdk etsikyvshq hpshpqlfsi
121 vrqacvrsls cevcpgregp iffgdeqhgf vfshtffikd slargfqrwy siitimmdri
181 ylinswpfll gkvrgiidel qgkalkvfea eqfgcpqraq rmntaftpfl hqrngnaars
241 ltsltsddnl waclhtsfaw llkacgsrlt ekllegapte dtivqmekla dleeeseswd
301 nseaeeeeka pvlpestegr eltqgpaess slsgcgswqp rklpvfkslr hmrqvlgaps
361 frmlawhvlm gnqviwksrd vdlvqsafev lrtmlpvgcv riipyssqye eayrcnflgl
421 sphvqipphv lssefavive vhaaarstlh pvgceddqsl skyefvvtsg spvaadrvgp
481 tilnkieaal tnqnlsvdvv dqclvclkee wmnkvkvlfk ftkvdsrpke dtqkllsilg
541 aseednvkll kfwmtglskt ykshlmstvr sptasesrn
```

The challenge of specifically targeting hepatocytes with RNAi methods has largely been solved, as evidenced by the existence of multiple clinically approved agents (for example, ONPATTRO® (patisiran), produced by Alnylam Pharmaceuticals, Inc.).

Alternatively, in the present compositions and methods, CRIPSR using a gRNA that specifically binds to, recognizes, or hybridizes to FLCN can be used to downregulate and/or silence the FLCN gene. In one embodiment, the gRNA binds to, recognizes, or hybridizes to the nucleic acid encoding the amino acid sequence of SEQ ID NO: 1.

The presently disclosed compositions and methods embody liver targeted inhibitors of folliculin, e.g., targeting of FLCN, as a novel therapy for treating NAFLD and NASH. The present compositions and methods represent an advantageous and new approach in multiple respects, including: (1) direct targeting to liver, thus minimizing side effects of systemic therapy (e.g., Intercept, a FXR small molecule agonist developed by Gilead Sciences, Inc., has been plagued by itching-induced medication discontinuations); (2) robust inhibition and reversal of both NAFLD and NASH; (3) intelligent mTOR inhibition, i.e., FLCN inhibits a key nodal point of metabolic regulation in liver, leading to coordinated metabolic reprogramming without counterregulatory compensation (e.g., ACC inhibitors, aimed at inhibition only fatty acid oxidation, have been plagued by compensatory activation of lipogenesis and hypertriglyceridemia).

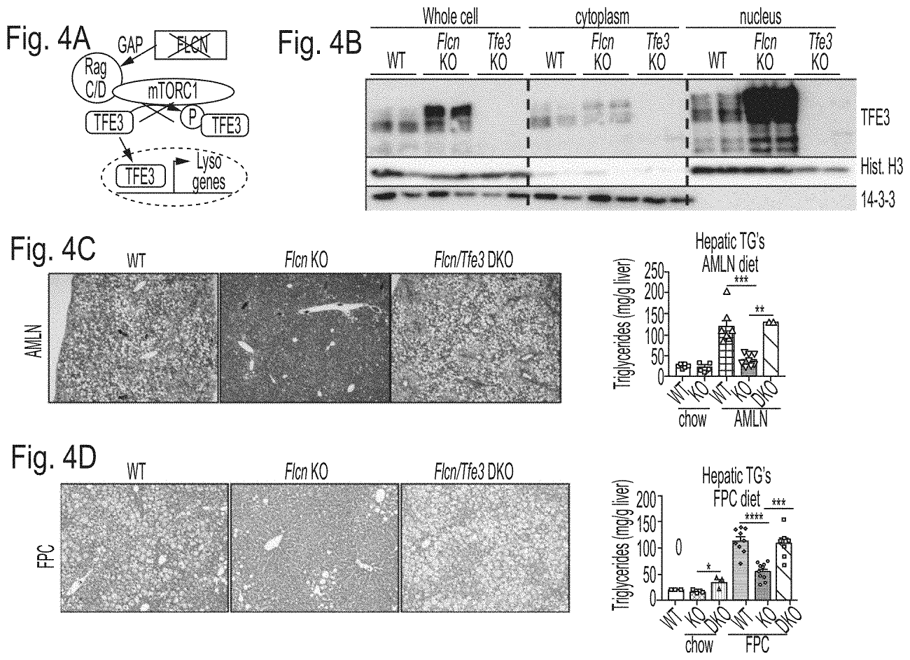

Thus, the present inventors have identified a novel target for treatment and prevention of NAFLD and NASH, i.e., folliculin. The protein folliculin (the product of the gene FLCN) is a RAG GTPase activating protein (GAP) for the mTORC1 complex (FIG. 4A). FLCN is expressed abundantly in the human liver. Mechanistically, FLCN activates mTORC1 to phosphorylate, and promote cytoplasmic sequestration of, the transcription factor E3 (TFE3), a key driver of lysosomal and mitochondrial biogenic programs. FLCN deletion induces TFE3 nuclear localization (FIG. 4A). Canonical mTORC1 signaling (e.g., S6K) is left intact in the absence of FLCN.

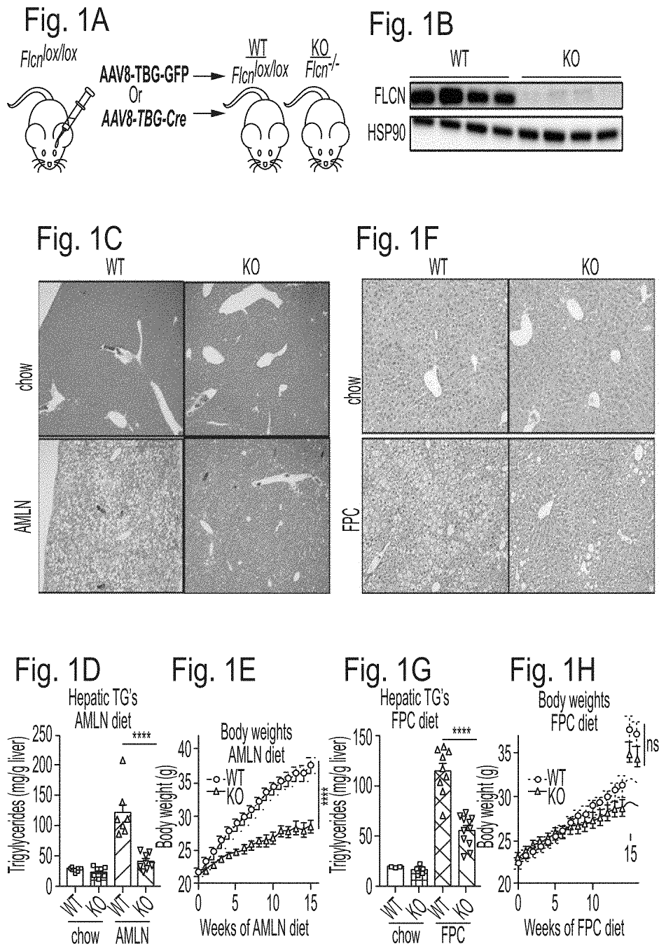

As described in the examples below, the present inventors developed a mouse model in which deletion of FLCN can be induced specifically in hepatocytes. Mice genetically bearing floxed Flcn alleles are acutely infected with AAV8-TBG-Cre. AAV8 has tropism for hepatocytes, and the TBG promoter assures expression of Cre only in hepatocytes; consequently, FLCN is deleted only in hepatocytes (FIG. 1A-1B). The effects of deletion of FLCN were tested on two different diet-induced models of NAFLD: 1. High-fat/high-fructose diet, plus fructose/glucose in the water, mimicking the American “Big Mac and Coke” diet; and 2. High-fat/high-fructose/high-cholesterol diet (AMLN diet), widely used as an aggressive model of NAFLD; In both cases, inducible deletion of FLCN in hepatocytes completely prevented NAFLD, as assayed by histology and triglyceride content (FIGS. 1C, 1D, 1G). Importantly, the mice were otherwise healthy, growing normally, and showing no signs of toxicity. Equally importantly, plasma triglycerides were not elevated after deletion of FLCN—an effect which has haunted other NAFLD drug candidates, such as ACC1/2 inhibitors.

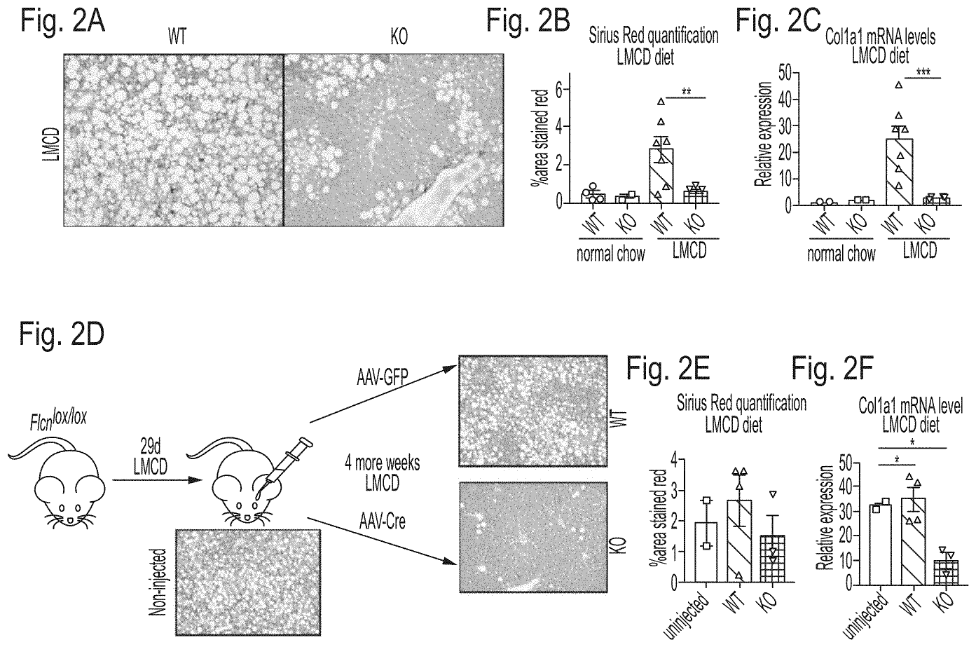

NAFLD is a precursor of NASH. The effects of deletion of FLCN on NASH were tested by using a low methionine/choline deficient/high fat diet (LMCD diet), widely used to induce both NAFLD and NASH. Inducible deletion of FLCN in hepatocytes completely prevented NASH in this model, as assayed by histology, Sirius red staining, and quantification of Col1a1 mRNA expression, a widely used marker for fibrosis (FIGS. 2A, 2C).

The ideal pharmaceutical would be able not only to prevent NAFLD/NASH, but also to reverse it after it has developed. To test this possibility, AAV-Cre was injected after 4 weeks of LMCD feeding and the development of NAFLD and NASH. Inducible deletion of FLCN at this time completely reversed NASH in this model, as assayed by histology, Sirius red staining, and quantification of Col1a1 mRNA expression (FIGS. 2D-2F).

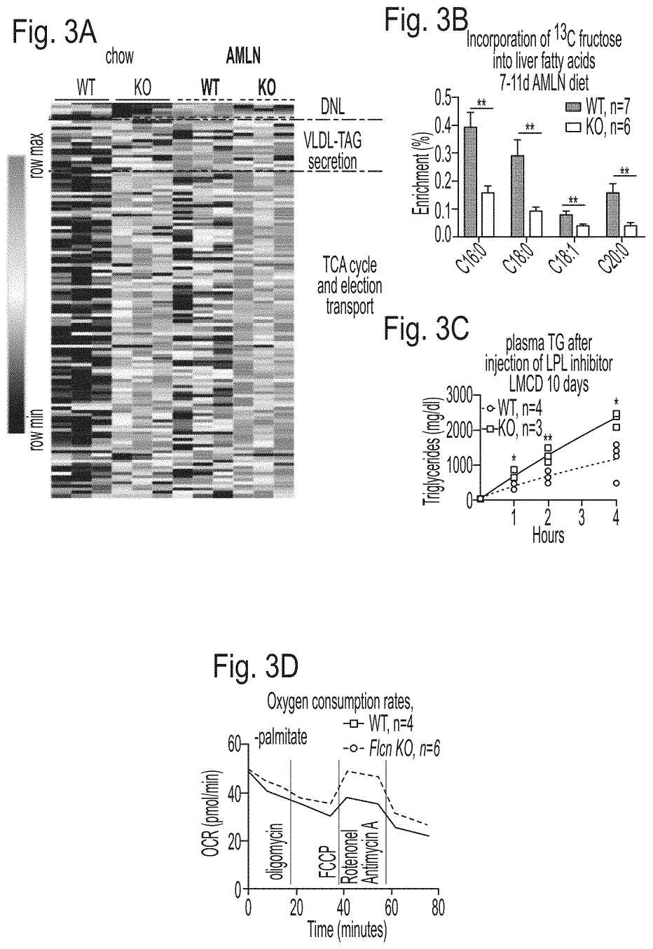

FLCN in large part suppresses TFE3 activity in the nucleus. The beneficial effects of deleting FLCN in the liver are entirely dependent on unleashing TFE3 activity, because codeletion of TFE3 in vivo completely blocks the suppression of NAFLD seen with FLCN deletion (FIGS. 4C, 4D). As noted above, canonical mTORC1 signaling is unaffected (not shown). FLCN deletion unleashes TFE3 to protect against NAFLD via multiple mechanisms: (1) suppression of de novo lipogenesis in the liver, as measured by incorporation of heavy-labeled fructose into liver fatty acids; (2) stimulation of secretion of triglycerides (VLDLTAG) from the liver, as measured by plasma TAGs after injection of an LPL inhibitor; and (3) stimulation of fatty acid oxidation, as measured by oxygen consumption rates in isolated hepatocytes (FIGS. 3B-3D).

In summary, it was discovered that induced suppression of FLCN in the liver efficiently protects from, and reverses, NAFLD and NASH, in multiple diet-induced rodent models, and does so via a synchronized process of both suppressing lipid synthesis and activating lipid disposal. Additional details are provided in the Examples, infra.

In accordance with the present methods, the inhibitor of folliculin administered in a therapeutically effective amount. As used herein, the phrase “therapeutically effective amount” refers to the amount of active compound that elicits the biological or medicinal response that is being sought in a tissue, system, animal, individual or human by a researcher, veterinarian, medical doctor or other clinician, which includes one or more of the following:

(1) at least partially preventing the disease or condition or a symptom thereof; for example, preventing a disease, condition or disorder in an individual who may be predisposed to the disease, condition or disorder but does not yet experience or display the pathology or symptomatology of the disease;

(2) inhibiting the disease or condition; for example, inhibiting a disease, condition or disorder in an individual who is experiencing or displaying the pathology or symptomatology of the disease, condition or disorder (i.e., including arresting further development of the pathology and/or symptomatology); and

(3) at least partially ameliorating the disease or condition; for example, ameliorating a disease, condition or disorder in an individual who is experiencing or displaying the pathology or symptomatology of the disease, condition or disorder (i.e., including reversing the pathology and/or symptomatology).

The inhibitor of folliculin may be provided in a composition that is formulated for any type of administration. For example, the compositions may be formulated for administration orally, topically, parenterally, enterally, or by inhalation. The inhibitor of folliculin may be formulated for neat administration, or in combination with conventional pharmaceutical carriers, diluents, or excipients, which may be liquid or solid. The applicable solid carrier, diluent, or excipient may function as, among other things, a binder, disintegrant, filler, lubricant, glidant, compression aid, processing aid, color, sweetener, preservative, suspensing/dispersing agent, tablet-disintegrating agent, encapsulating material, film former or coating, flavoring agent, or printing ink. Any material used in preparing any dosage unit form is preferably pharmaceutically pure and substantially non-toxic in the amounts employed. In addition, the inhibitor of folliculin may be incorporated into sustained-release preparations and formulations. Administration in this respect includes administration by, inter alia, the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol, and rectal systemic.

In powders, the carrier, diluent, or excipient may be a finely divided solid that is in admixture with the finely divided active ingredient. In tablets, the active ingredient is mixed with a carrier, diluent or excipient having the necessary compression properties in suitable proportions and compacted in the shape and size desired. For oral therapeutic administration, the active compound may be incorporated with the carrier, diluent, or excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained.

Liquid carriers, diluents, or excipients may be used in preparing solutions, suspensions, emulsions, syrups, elixirs, and the like. The active ingredient of this invention can be dissolved or suspended in a pharmaceutically acceptable liquid such as water, an organic solvent, a mixture of both, or pharmaceutically acceptable oils or fat. The liquid carrier, excipient, or diluent can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators.

Suitable solid carriers, diluents, and excipients may include, for example, calcium phosphate, silicon dioxide, magnesium stearate, talc, sugars, lactose, dextrin, starch, gelatin, cellulose, methyl cellulose, ethylcellulose, sodium carboxymethyl cellulose, microcrystalline cellulose, polyvinylpyrrolidine, low melting waxes, ion exchange resins, croscarmellose carbon, acacia, pregelatinized starch, crospovidone, HPMC, povidone, titanium dioxide, polycrystalline cellulose, aluminum methahydroxide, agar-agar, tragacanth, or mixtures thereof.

Suitable examples of liquid carriers, diluents and excipients, for example, for oral, topical, or parenteral administration, include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil), or mixtures thereof.

For parenteral administration, the carrier, diluent, or excipient can also be an oily ester such as ethyl oleate and isopropyl myristate. Also contemplated are sterile liquid carriers, diluents, or excipients, which are used in sterile liquid form compositions for parenteral administration. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier, diluent, or excipient may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating inhibitor of Folliculin in the pharmaceutically appropriate amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and freeze drying techniques that yield a powder of the active ingredient or ingredients, plus any additional desired ingredient from the previously sterile-filtered solution thereof.

Thus, the inhibitor of folliculin may be administered in an effective amount by any of the conventional techniques well-established in the medical field. For example, the administration may be in the amount of about 0.01 mg/day to about 500 mg per day. In some embodiments, the administration may be in the amount of about 250 mg/kg/day. Thus, administration may be in the amount of about 0.01 mg/day, 0.02, mg/day, 0.05 mg/day, 0.07 mg/day, 0.1 mg/day, about 0.5 mg/day, about 1.0 mg/day, about 5 mg/day, about 10 mg/day, about 20 mg/day, about 50 mg/day, about 100 mg/day, about 200 mg/day, about 250 mg/day, about 300 mg/day, or about 500 mg/day.

In one embodiment, the siRNA and/or gCas9 enzyme and a guide RNA (gRNA) that specifically binds to, recognizes, or hybridizes to FLCN are formulated in a lipid nanoparticle. Accordingly, one embodiment of the invention is directed to intracellular delivery of the siRNA and gRNA of the disclosure. In one embodiment, the siRNA and/or gCas9 enzyme and a guide RNA (gRNA) that specifically binds to, recognizes, or hybridizes to FLCN are formulated in the lipid nanoparticle.

### CRISPR/Cas

In another embodiment, the invention is directed methods of preventing or treating nonalcoholic fatty liver disease or nonalcoholic steatohepatitis in a subject comprising silencing or down-regulating FLCN in hepatocytes of the subject. This may be achieved using genome editing technology to silence or reduce FLCN expression. In certain embodiments, FLCN is silenced or downregulated using an RNA-guided nuclease. The RNA-guided nuclease is a CRISPR-Cas9 combination comprising a Cas9 enzyme and a guide RNA (gRNA) that specifically binds to, recognizes, or hybridizes to a FLCN (i.e. a part of the FLCN gene). In one embodiment, the guide RNA (gRNA) that specifically binds to, recognizes, or hybridizes to a part of the nucleic acid sequence of FLCN transcript variant 1-5 (SEQ ID NO: 5-9). In one embodiment, the gRNA binds to, recognizes, or hybridizes to a part of the nucleic acid sequence encoding the amino acid sequence of SEQ ID NO: 1. In another embodiment, the gRNA comprises a nucleic acid of SEQ ID NO: 2-4. In alternate embodiments, the gRNA consists or consists essentially of a nucleic acid of SEQ ID NO: 2-4. In another embodiment, the gRNA comprises a nucleic acid that is at least 85%, at least 87%, at least 90%, at least 95%, at least 96%, or at least 98% identical to the nucleic acid sequence of SEQ ID NO: 2-4.

In one embodiment, the gRNA specifically binds to, recognizes, or hybridizes to the FLCN gene or fragment thereof. In another embodiment, the gRNA specifically binds to, recognizes, or hybridizes a nucleic acid encoding the folliculin protein.

The CRISPR/Cas system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/CAS system can thereby be engineered to cleave virtually any DNA sequence by redesigning the gRNA in cell lines (such as 293T cells), primary cells, and CAR T cells. The CRISPR/CAS system can simultaneously target multiple genomic loci by co-expressing a single CAS9 protein with two or more gRNAs, making this system uniquely suited for multiple gene editing or synergistic activation of target genes.

One example of a CRISPR/Cas system used to inhibit gene expression, CRISPRi, is described in U.S. Publication No.: 2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In one embodiment, the CRISPR system comprises an expression vector, such as, but not limited to, an pAd5F35-CRISPR vector. In one embodiment, the modified T cell described herein is further modified by introducing a Cas expression vector and a guide nucleic acid sequence specific for a gene into the modified T cell. In another embodiment, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

The guide nucleic acid sequence is specific for a gene and targets that gene for Cas endonuclease-induced double strand breaks. The sequence of the guide nucleic acid sequence may be within a loci of the gene. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length. The guide RNA (gRNA) specifically binds to, recognizes, or hybridizes to the FLCN gene, fragment thereof, or a nucleic acid encoding the folliculin protein (SEQ ID NO: 1).

### Introduction of Nucleic Acids

In certain embodiments, the method relies on known methods of introducing nucleic acids to administer the siRNA and/or gCas9 enzyme and a guide RNA (gRNA) that specifically binds to, recognizes, or hybridizes to FLCN.

Methods of introducing nucleic acids into a cell include physical, biological, and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001)).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Moreover, the siRNA and/or gCas9 enzyme and a guide RNA (gRNA) that specifically binds to, recognizes, or hybridizes to FLCN may be introduced/administered by any means.

The disclosure also provides for vectors containing the siRNA or gRNA of the disclosure. The vector can have a nucleic acid sequence containing an origin of replication. The vector can be a plasmid, bacteriophage, bacterial artificial chromosome, or yeast artificial chromosome. The vector can be either a self-replicating extrachromosomal vector or a vector which integrates into a host genome.

Furthermore, the disclosure incudes kits containing the siRNA or a CRISPR-Cas9 combination comprising a Cas9 enzyme and a guide RNA (gRNA) that specifically binds to, recognizes, or hybridizes to FLCN. The kit can further include instructions or a label for using the kit to prevent or treat nonalcoholic fatty liver disease or nonalcoholic steatohepatitis.

### EXAMPLES

The following examples are set forth so as to provide those of ordinary skill in the art with a complete disclosure and description of how the formulations, methods, and articles claimed herein may be developed and evaluated, and are intended to be purely exemplary of the invention and are not intended to limit the scope of what the inventors regard as their invention. Efforts have been made to ensure accuracy with respect to numbers (e.g., amounts), but some errors and deviations should be accounted for.

### Example 1—FLCN Deletion Protects Against NAFLD

FIG. 1A-1H provide the results of a study regarding the results of deleting the FLCN gene using AAV8-TBG-GFP or AAV8-TBG-Cre. FLCN deletion protects against non-alcoholic fatty liver disease (NAFLD) (FIGS. 1A-1B) Flcnlox/lox mice were injected with hepatocyte-specific AAV8-TBG-GFP or AAV8-TBG-Cre to yield wild-type “WT” and hepatocyte-specific Flcn loss-of-function “KO” (knock-out) mice. Mice were challenged with either normal chow or the AMLN diet, a NAFLD-inducing diet that is high in cholesterol, fat, and fructose. As shown in FIG. 1C, liver H&E staining reveals severe steatosis in WT mice treated with the AMLN diet, whereas KO mice were highly protected against NAFLD. As shown in FIG. 1D, hepatic triglyceride quantification confirmed the findings with histology. As shown in FIG. 1E, KO mice were protected against body-weight gain with the AMLN diet. As shown in FIGS. 1F-G, mice were challenged with either normal chow or a second NAFLD-inducing diet (the FPC diet), which is high in cholesterol, fat, sucrose, low in choline and Vitamin E, and also containing fructose and glucose in the drinking water. Liver H&E staining and triglyceride quantification revealed that KO mice were protected against steatosis in this second diet. In FIG. 1H, body weight measurements revealed steatosis protection is irrespective of body weight changes. ****p<0.0001, ns=not significant, with student's unpaired two-tailed t-test.

### Example 2—FLCN Deletion Protects Against and Reverses Non-Alcoholic Steatohepatitis (NASH)

WT and KO mice were challenged with either normal chow or the LMCD diet, a NASH-inducing diet that is high in fat, low in methionine, and choline-deficient. Liver Sirius Red staining revealed severe steatosis and fibrosis in WT mice treated with the LMCD diet, whereas KO mice were highly protected against these phenotypes. Results are shown in FIGS. 2A and 2B. As shown in FIG. 2C, Col1a1 mRNA, a key marker of fibrosis, was suppressed in KO mice, consistent with a protection against NASH. Non-injected Flcnlox/lox mice were challenged with 29 days of LMCD diet to induce NASH, as revealed by Sirius Red staining. The mice were then injected with AAV8-TBG-GFP or AAV8-TBG-Cre and challenged with another four weeks of LMCD diet. Mice were then sacrificed, and livers harvested. As shown in FIGS. 2D-2F, GFP-injected “WT” livers still displayed NASH via Sirius Red liver staining and Col1a1 mRNA measurement after the additional four weeks of diet, but Cre-injected “KO” livers were histologically normal with suppressed Col1a1 mRNA, thereby indicating Flcn deletion is able to reverse NASH. Sirius Red quantification trended towards statistical significance. *p<0.05, **p<0.01, ***p<0.001, with student's unpaired two-tailed t-test.

### Example 3—FLCN Coordinates Several Hepatic Lipid Processes, and FLCN Deletion Unleashes TFE3 to Protect Against NAFLD Via Multiple Mechanisms

RNA sequencing was performed on WT and KO livers from mice treated with normal chow or AMLN diet. Results (FIG. 3A) revealed down-regulation of de novo lipogenesis (DNL) genes, but up-regulation of genes for very low density lipoprotein-triacylglycerol (VLDL-TAG) secretion as well as up-regulation of oxidative genes, suggesting Flcn deletion causes decreased synthesis of lipids as well as increased clearance. WT and KO mice on a short term AMLN diet were given a bolus of isotopically labeled 13C fructose. 13C label was then traced into hepatic fatty acids as a read-out of de novo lipogenesis. Decreased incorporation of label in KO livers indicated suppressed de novo lipogenesis (FIG. 3B). WT and KO mice on a short-term LMCD diet were given a bolus of Poloxamer 407, an inhibitor of lipoprotein lipase (LPL). Plasma triglycerides were then serially measured as a read-out of hepatic VLDL-TAG secretion. As provided in FIG. 3C, KO mice displayed higher plasma triglycerides, indicative of increased VLDL-TAG secretion. *p<0.05, **p<0.01, with student's unpaired two-tailed t-test. FIG. 3D illustrates that TFE3 protects against NAFLD by stimulation of fatty acid oxidation, as measured by oxygen consumption rates in isolated hepatocytes.

### Example 4—Flcn KO Mice are Protected from Steatosis Via TFE3 Activation

FIG. 4A provides a schematic of the FLCN/mTORC1/TFE3 pathway. FLCN activates RagC, which consequently allows mTORC1 to phosphorylate and cytoplasmically sequester the transcription factor TFE3. Deletion of FLCN inhibits this process and TFE3 subsequently localizes to the nucleus where it can transcribe its target genes. Livers from WT, KO, and Tfe3 whole-body knock-out (Tfe3^{-/Y}) mice were subjected to subcellular fractionation followed by Western Blot. As shown in FIG. 4B, TFE3 protein was highly nuclear in KO livers as expected. To yield Flcn/Tfe3 double knock-out (“DKO”) mice, double-mutant mice were made harboring Flcn^{lox/lox} alleles as well as a whole-body deletion of Tfe3 (Tfe3^{-/Y}). These mice were subsequently injected with AAV8-TBG-Cre to produce DKO mice. As shown in FIGS. 4C and 4D, when challenged with both the AMLN and FPC diets, Flcn/Tfe3 DKO mice revealed rampant steatosis similar to that of WT mice, suggesting that TFE3 activation is key to protection against steatosis in Flcn-null mice. *p<0.05, **p<0.01, ***p<0.001, ****p<0.0001 with one-way ANOVA.

### Example 5: FLCN Protein is Decreased in AML12's Infected with Lentivirus Containing Various Flcn Guide RNA

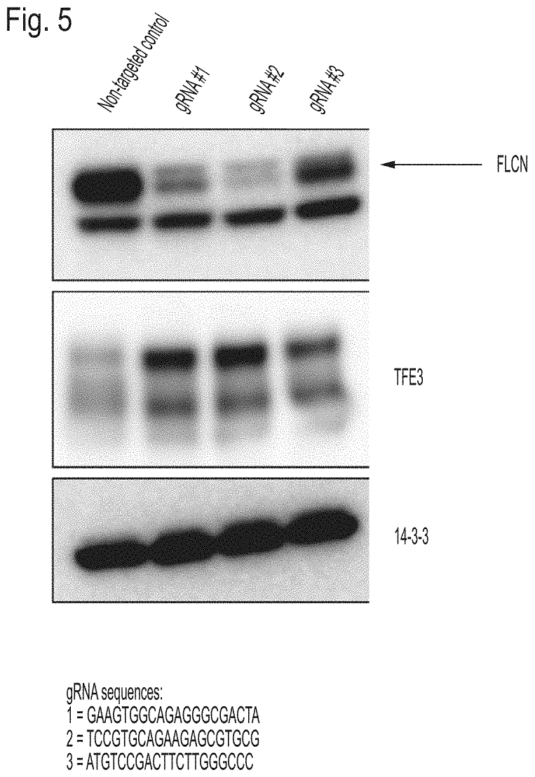

Flcn guide RNAs were constructed and cloned into the lentiCRISPR v2 puro backbone. AML12 (hepatocytes) were infected with a lentivirus containing three different guide RNA molecules binding to FLCN as follows: gRNA #1 (GAAGTGGCAGAGGGCGACT (SEQ ID NO: 2)); gRNA #2 (TCCGTGCAGAAGAGCGTGCG (SEQ ID NO: 3)); and gRNA #3 (ATGTCCGACTTCTTGGGCCC (SEQ ID NO: 4)). Using CRISPR, FLCN was targeted using the gRNAs and the gene expression was inhibited. After CRISPR, the levels of FLCN, FTE and 14-3-3 were assessed using Western Blot. Flcn-null cells exhibit classic high molecular weight band in TFE3 protein, characteristic of nuclear TFE3. Compared to the non-targeted control (see FIG. 5), the AML cells treated with the three gRNAs exhibited a decrease in FLCN protein. The cells treated with the gRNA also exhibited a high molecular band in TFE which is known to be characteristic of nuclear TFE3.

### ILLUSTRATIVE EMBODIMENTS

Provided here are illustrative embodiments of the disclosed technology. These embodiments are illustrative only and do not limit the scope of the present disclosure or of the claims attached

Embodiment 1a. A composition for preventing or treating nonalcoholic fatty liver disease or nonalcoholic steatohepatitis in a subject comprising an inhibitor of folliculin.

Embodiment 2a. The composition according to embodiment 1a, wherein the inhibitor of folliculin is targeted to hepatocytes of the subject.

Embodiment 3a. The composition according to embodiment 1a or embodiment 2a, wherein the inhibitor of folliculin operates by RNA interference.

Embodiment 4a. The composition according to any one of the preceding embodiments, wherein the inhibitor of folliculin is a GalNac-modified oligonucleotide.

Embodiment 5a. The composition according to any one of the preceding embodiments, wherein the inhibitor of folliculin is small interfering RNA (siRNA).

Embodiment 6a. A method for preventing or treating nonalcoholic fatty liver disease or nonalcoholic steatohepatitis in a subject comprising administering to the liver of the subject an inhibitor of folliculin.

Embodiment 7a. The method according to embodiment 6a, wherein the inhibitor of folliculin is targeted to hepatocytes of the subject.

Embodiment 8a. The method according to embodiment 6a or embodiment 7a, wherein the inhibitor of folliculin operates by RNA interference.

Embodiment 9a. The method according to any one of embodiments 6a-8a, wherein the inhibitor of folliculin is a GalNac-modified oligonucleotide.

Embodiment 10a. The method according to any one of embodiments 6a-9a, wherein the inhibitor of folliculin is small interfering RNA (siRNA).

Embodiment 11a. A method for preventing or treating nonalcoholic fatty liver disease or nonalcoholic steatohepatitis in a subject comprising silencing or down-regulating FLCN in hepatocytes of the subject.

Embodiment 12a. The method according to embodiment 11a, wherein FLCN is silenced in hepatocytes of the subject by RNA interference.

Embodiment 13a. The method according to embodiment 11a, wherein FLCN is silenced in hepatocytes of the subject using a GalNac-modified oligonucleotide.

Embodiment 14a. The method according to embodiment 11a, wherein FLCN is silenced in hepatocytes of the subject using small interfering RNA (siRNA).

Nucleic Acid Sequence of FLCN transcript variant 1 (SEQ ID NO: 5):

```
LOCUS New_DNA 3667 bp ds-DNA linear
COMMENT ApEinfo:methylated:1
ORIGIN
      1 ggcggggctg cgggaccgcg agtgagtgtg gtcgctcctg gttctgccag ctcccctgag
    61 agcctgaacc cgggcttgag agcctcgcca ccccgggtga catccctgcc gtgggcttgg
  121 gggctctggg tgtgattccg ccggtccggg tcccgcagcg accacctacc cagcgcagtc
  181 aggggcgggg ctgggaccca gagcgggacc ccggctgccg agtccaggtg tcccgcgggc
  241 ctcgatttgg ggagcagaaa acgccaggtc ttcaagggtg tctgccacca ccatgcctga
  301 cccatttggc agcagcctcg tgtgtggtgg tctggtgtgg acggtggaag cgtgattctg
  361 ctgagtgtca gtgtgaccac tcgtgctcag ccgtatctca gcaggaggac aggtgccgga
  421 gcagctcgtg cagctaagca gccaactgca gaaacgtcag gcctgttgca gtctccaagg
  481 caccatgaat gccatcgtgg ctctctgcca cttctgcgag ctccacggcc cccgcactct
  541 cttctgcacg gaggtgctgc acgccccact tcctcaaggg gatgggaatg aggacagtcc
  601 tggccagggt gagcaggcgg aagaagagga aggtggcatt cagatgaaca gtcggatgcg
  661 tgcgcacagc cccgcagagg gggccagcgt cgagtccagc agcccggggc ccaaaaagtc
  721 ggacatgtgc gagggctgcc ggtcacttgc tgcagggcac ccgggatata tcagccatga
  781 taaagagacc tccattaaat acgtcagcca ccagcacccc agccaccccc agctcttcag
  841 cattgtccgc caggcctgtg tccggagcct gagctgtgag gtctgccctg gccgtgaagg
  901 ccccatcttc ttcggagatg agcagcacgg ctttgtgttc agccacacct tcttcatcaa
  961 ggacagcctg gccaggggct tccagcgctg gtacagcatc atcaccatca tgatggaccg
1021 gatctacctc atcaactcct ggcccttcct gctggggaag gtccggggaa tcatcgatga
1081 gctccagggc aaggcgctca aggtgtttga ggcagagcag tttggatgcc cacagcgtgc
1141 tcagaggatg aacacagcct tcacgccatt cctacaccag aggaacggca acgccgcccg
1201 ctcgctgaca tcgctgacaa gtgatgacaa cctgtgggcg tgcctgcaca cctcctttgc
1261 ctggctcctg aaggcgtgtg gcagccggct gaccgagaag ctcctggaag gtgctccgac
1321 cgaggatacc ttggtccaga tggagaagct cgctgattta gaagaggaat cagaaagctg
1381 ggacaactct gaggctgaag aggaggagaa agcccctgtg ttgccagaga gtacagaagg
1441 gcgggagctg acccagggcc cggcagagtc ctcctctctc tcaggctgtg ggagctggca
1501 gccccggaag ctgccagtct tcaagtccct ccggcacatg aggcaggtcc tgggtgcccc
1561 ttctttccgc atgctggcct ggcacgttct catggggaac caggtgatct ggaaaagcag
1621 agacgtggac ctcgtccagt cagcttttga agtacttcgg accatgcttc ccgtgggctg
1681 cgtccgcatc atcccataca gcagccagta cgaggaggcc tatcggtgca acttcctggg
1741 gctcagcccg cacgtgcaga tcccccccca cgtgctctcc tcagagtttg ctgtcatcgt
1801 ggaggtccac gcagccgcac gttccaccct ccaccctgtg gggtgtgagg atgaccagtc
1861 tctcagcaag tacgagtttg tggtgaccag tgggagccct gtagctgcag accgagtggg
1921 ccccaccatc ctgaataaga ttgaagcggc tctgaccaac cagaacctgt ctgtggatgt
1981 ggtggaccag tgcctcgtct gcctcaagga ggagtggatg aacaaagtga aggtgctttt
2041 taagttcacc aaggtggaca gtcgacccaa agaggacaca cagaagctgc tgagcatcct
2101 gggtgcgtcc gaggaggaca atgtcaagct gctgaagttc tggatgactg gcctgagcaa
2161 gacctacaag tcacacctca tgtccacggt ccgcagcccc acagcctcgg agtctcggaa
2221 ctgacccgtc acacacacct gcctaaagac agggatggct gtccacagga tcctccagcc
2281 ccgtgagagg gactgtccct tgagtttctc aactgctgga aggagctgtg tcccagcaag
2341 gaagggaaac catcagggct gggctcggcc ctgtcaggtt tggggcctgt gtgcttccca
2401 gactctccct ccagccgttg gaatcgctga agatggcaat gaaaggcgga gggatgatgg
2461 gctctctctg tgttcaaact ccttggagag acgactagga ggacagcttg cctcccaggc
2521 cccttgtgga cttagactca aaacccgcag gagaaacagg tccgactcag tatgcagtcg
2581 caataacatg tctgctcccg aggttaacat tcaagcgttt ctactttgaa attcagcaag
2641 agtttctggg ccttatgttt gagggtacct tttgctgcag ttgtgaatat tcagtacatt
2701 gccagctctt ggtcactgag tgattgagtt agggctccgc aagagacttt ggggagtgaa
2761 gtggatctct tcctcatctt ttggtcctct gaaatgtgtg ttctgaagcc atggggctcg
2821 tcttctgggg tgttcccctg caggtgctgg tgaaggtaac ctggggctta atgatggagt
2881 ccctgatcat ttttgcacaa gacaggttgc tgaggggtcg gcaagcatct gacttgccca
2941 atcccctgga tatggtgagc cccgccatgc ttttattctg tatcgctttt gtctttattg
3001 ctgctttcaa catttacgtt tggttacagt taactatttt cggagtgtgg tgattgaaga
3061 caatttcatc atcccactgt actttttttt ttgagaggga gtttcactct tgttgcccag
3121 gctggagtgc aatggcacga tcttggctca ctgcaacctc tgcctcctgg gttcaagcaa
3181 ttctcctgcc tcagcctcca gagtagctgg aactacaggt gcccgccact atgcccagct
3241 aatttttgta ttttttagta gagacggggt ttcaccgtgt tggccgggct ggtctcaaac
3301 tcctgacctc aggtgatcca cccacctcag cctcccaaag tgctgggatt acaagcgtga
3361 gccactgtgc ctggcccttt tttttttttt tttttttttt tttaaagaga tggcatcttg
3421 ctatgtcgtc caggctggtc ttgaactcct gagttcaagc agtcctcctg cttcaacata
3481 cagctacagg taccccccac tatacatttt taataaggat tcatggctca gagggatttt
3541 ctgatggttt tgctgatttg tttctagttt ttttgtgttt atatttaaca tgaagaccaa
3601 gtttatataa ctaggtatct gtataatgca acaacattgg aacacaataa agatgtattt
3661 ttgtaaa
//
```

Nucleic Acid Sequence of FLCN transcript variant 2 (SEQ ID NO: 6):

```
LOCUS New_DNA 3325 bp ds-DNA
COMMENT ApEinfo:methylated:1
ORIGIN
      1 ggcggggctg cgggaccgcg agtgagtgtg gtcgctcctg gttctgccag ctcccctgag
    61 agcctgaacc cgggcttgag agcctcgcca ccccgggtga catccctgcc gtgggcttgg
  121 gggctctggg tgtgattccg ccggtccggg tcccgcagcg accacctacc cagcgcagtc
  181 aggggcgggg ctgggaccca gagcgggacc ccggctgccg agtccaggtg tcccgcgggc
  241 ctcgatttgg ggagcagaaa acgccaggtc ttcaagggtg tctgccacca ccatgcctga
  301 cccatttggc agcagcctcg tgtgtggtgg tctggtgtgg acggtggaag cgtgattctg
  361 ctgagtgtca gtgtgaccac tcgtgctcag ccgtatctca gcaggaggac aggtgccgga
  421 gcagctcgtg cagctaagca gccaactgca gaaacgtcag gcctgttgca gtctccaagg
  481 caccatgaat gccatcgtgg ctctctgcca cttctgcgag ctccacggcc cccgcactct
  541 cttctgcacg gaggtgctgc acgccccact tcctcaaggg gatgggaatg aggacagtcc
  601 tggccagggt gagcaggcgg aagaagagga aggtggcatt cagatgaaca gtcggatgcg
  661 tgcgcacagc cccgcagagg gggccagcgt cgagtccagc agcccggggc ccaaaaagtc
  721 ggacatgtgc gagggctgcc ggtcacttgc tgcagggcac ccgggatata tcagccatga
  781 taaagagacc tccattaaat acgtcagcca ccagcacccc agccaccccc agctcttcag
  841 cattgtccgc caggcctgtg tccggagcct gagctgtgag gtctgccctg gccgtgaagg
  901 ccccatcttc ttcggagatg agcagcacgg ctttgtgttc agccacacct tcttcatcaa
  961 ggacagcctg gccaggggct tccagcgctg gtacagcatc atcaccatca tgatggaccg
1021 gatctacctc atcaactcct ggcccttcct gctggggaag gtccggggaa tcatcgatga
1081 gctccagggc aaggcgctca aggtgtttga ggcagagcag tttggatgcc cacagcgtgc
1141 tcagaggatg aacacagcct tcacgccatt cctacaccag aggaacggca acgccgcccg
1201 ctcgctgaca tcgctgacaa gtgatgacaa cctgtgggcg tgcctgcaca cctcctttgc
1261 ctggctcctg aaggcgtgtg gcagccggct gaccgagaag ctcctggaag gtgctccgac
1321 cgaggatacc ttggtccaga tggagaagct cgctggtgag gcaggggtgc tgttgccggg
1381 gccttggccc ggatggccgt ggggcggtac cagctgtctg ctctcctggc aggaatcgct
1441 gagggaggga aacgcggctc tgaatcagcc cagaacgagc cttcgggaag ctcaccctcc
1501 gatctcggtg tgattgttgt gattgttgtg atttcctgtc tcgtttgcct tgaccgccat
1561 gtgaaagaat ctgttcccca gctaggtggg gaaaattcac aggtgggctg tctgtagaga
1621 gaactggctg attaaaggct tctcgtcccg attttgtgat agccaagtgc ttggcctggt
1681 cgacggtctt tgctccttta caaataaagt gttctgtttc agttcgtccc aagttttcca
1741 tgaagggcag tggttccctg acctcccagg tgcctgggct tccccaggtt cctgatctgg
1801 ggcttggggc cctgtgtttg gggatcgtgg cactgtgtgc accagcctgg aagcactggg
1861 ccagtcttgg ccaagctttc catcagggat gatttgatct tggtgctaca ggtctgtggt
1921 acgaccattg ttccacacca catgtcatta ataatgcttc ccatgcttct gcttgcaaat
1981 gaccagcctt ccaaacagcc agagctgttt cgaggtgttt ctgcaggcag gtgcaggcgt
2041 gccctcaaat aagctttgcc aatggagtct cagcaagagc aaaacctggt caggaaagac
2101 aaagcctggg aatccacccc catgccctgc aggttggctg gccctggagc catttattat
2161 agtgctaatc atgtttctag gcaggtgcag atggcaaggg cagtgtcttg gtgagctttt
2221 tagcacgaag agccaggtct gtcgaagcct ttgtgagagc tggaaacgca ggtgtgctgg
2281 gcatgcgcag tatggggttt cgggctcagg gcttgccctt tggcatcaga cagacctggc
2341 ttcgcatcct ggatttgctt ctgacgtgca cccttccctt tgggtctcgt gatgtgaaat
2401 ggagatgttg tcatttgtga gggctccatg aagtttcgtt gaaatgacaa atactaattt
2461 cttcatctgt gaaatggaga taatagtgct gacctcagaa cagctgagag gactaaatga
2521 aatgatgttg gatgtagcca taaagaacga agtcaggcac tggtgcacgc ctggaatccc
2581 agctcttggg agaccgagac aggtggattg cttgagctca ggagtttgag accagcctga
2641 gcaacatagg gaggtccagt ctctacaaaa aatatgaaaa gtagctgggc gtggtggcgc
2701 atgcctgtag tcccactact tggaaggctt cgttgggagg atcacttgag cccagaagat
2761 tgaggctgca gtaagccgtg atcgtgccac tgcattccag cctgggcaac agagcgagac
2821 actgtctcaa ataaaaaaga tgggaatagt agacactggg ggctccagaa ggagggaggg
2881 agggaggaag gggaggaagg gctgaaatgc tttctattgg atactatctg ggcatattac
2941 ttcctgtggt tcactgtctg ggtgacagga ttcatagaag cccaaacttt agcaccacgc
3001 agcataccct tgtaacaaag ccgcacacgt acgccctcaa gctaaaacaa aagtggaccg
3061 ggaggccgag gtcgggggat catgaggtca ggagtttgag accagcctgg cagataacgg
3121 tgaaaccccg tctctactaa aaataccaaa aaaagttagc cggacatggt ggcaggtgcc
3181 tgtagtccca gctacttggg aggctggggc agaagaatcg cttgaaccca ggaggcggag
3241 gttgcagtga gccgagattg cgccactgca ctccagcctg tgcgacagag tgagactccg
3301 tctcaaaaaa aaaaaaaaaa aaaaa
//
```

Nucleic Acid Sequence of FLCN transcript variant 3 (SEQ ID NO: 7):

```
LOCUS New_DNA 3929 bp ds-DNA linear
COMMENT ApEinfo:methylated:1
ORIGIN
      1 ggcggggctg cgggaccgcg agtgagtgtg gtcgctcctg gttctgccag ctcccctgag
    61 agcctgaacc cgggcttgag agcctcgcca ccccgggtga catccctgcc gtgggcttgg
  121 gggctctggg tgtgattccg ccggtccggg tcccgcagcg accacctacc cagcgcagtc
  181 aggggcgggg ctgggaccca gagcgggacc ccggctgccg agtccaggtg tcccgcgggc
  241 ctcgatttgg ggagcaggtt ttgtcttcgc tctgtttgga ggagagggtg tgtgtcatcc
  301 tcttctccca gtttggcgtt caggagggtc ctctgatgcg ctaatagggt agcaccgtgt
  361 cctccaggga gggtggaaga ccgcgcttct ctccagttga gagtactgtc agtcgcgtcc
  421 ttgtctcctg gaaagaatgg attggcttgt ggattgaagt ccaagaaaac gccaggtctt
  481 caagggtgtc tgccaccacc atgcctgacc catttggcag cagcctcgtg tgtggtggtc
  541 tggtgtggac ggtggaagcg tgattctgct gagtgtcagt gtgaccactc gtgctcagcc
  601 gtatctcagc aggaggacag gtgccggagc agctcgtgca gctaagcagc caactgcaga
  661 aacgtcaggc ctgttgcagt ctccaaggca ccatgaatgc catcgtggct ctctgccact
  721 tctgcgagct ccacggcccc cgcactctct tctgcacgga ggtgctgcac gccccacttc
  781 ctcaagggga tgggaatgag gacagtcctg gccagggtga gcaggcggaa gaagaggaag
  841 gtggcattca gatgaacagt cggatgcgtg cgcacagccc cgcagagggg gccagcgtcg
  901 agtccagcag cccggggccc aaaaagtcgg acatgtgcga gggctgccgg tcacttgctg
  961 cagggcaccc gggatatatc agccatgata aagagacctc cattaaatac gtcagccacc
1021 agcaccccag ccacccccag ctcttcagca ttgtccgcca ggcctgtgtc cggagcctga
1081 gctgtgagtg cagggagagt gcagtggctg ctctgacctg ctggttcttc tgcatgctcc
1141 aggtctgccc tggccgtgaa ggccccatct tcttcggaga tgagcagcac ggctttgtgt
1201 tcagccacac cttcttcatc aaggacagcc tggccagggg cttccagcgc tggtacagca
1261 tcatcaccat catgatggac cggatctacc tcatcaactc ctggcccttc ctgctgggga
1321 aggtccgggg aatcatcgat gagctccagg gcaaggcgct caaggtgttt gaggcagagc
1381 agtttggatg cccacagcgt gctcagagga tgaacacagc cttcacgcca ttcctacacc
1441 agaggaacgg caacgccgcc cgctcgctga catcgctgac aagtgatgac aacctgtggg
1501 cgtgcctgca cacctccttt gcctggctcc tgaaggcgtg tggcagccgg ctgaccgaga
1561 agctcctgga aggtgctccg accgaggata ccttggtcca gatggagaag ctcgctgatt
1621 tagaagagga atcagaaagc tgggacaact ctgaggctga agaggaggag aaagcccctg
1681 tgttgccaga gagtacagaa gggcgggagc tgacccaggg cccggcagag tcctcctctc
1741 tctcaggctg tgggagctgg cagccccgga agctgccagt cttcaagtcc ctccggcaca
1801 tgaggcaggt cctgggtgcc ccttctttcc gcatgctggc ctggcacgtt ctcatgggga
1861 accaggtgat ctggaaaagc agagacgtgg acctcgtcca gtcagctttt gaagtacttc
1921 ggaccatgct tcccgtgggc tgcgtccgca tcatcccata cagcagccag tacgaggagg
1981 cctatcggtg caacttcctg gggctcagcc cgcacgtgca gatccccccc cacgtgctct
2041 cctcagagtt tgctgtcatc gtggaggtcc acgcagccgc acgttccacc ctccaccctg
2101 tggggtgtga ggatgaccag tctctcagca agtacgagtt tgtggtgacc agtgggagcc
2161 ctgtagctgc agaccgagtg ggccccacca tcctgaataa gattgaagcg gctctgacca
2221 accagaacct gtctgtggat gtggtggacc agtgcctcgt ctgcctcaag gaggagtgga
2281 tgaacaaagt gaaggtgctt tttaagttca ccaaggtgga cagtcgaccc aaagaggaca
2341 cacagaagct gctgagcatc ctgggtgcgt ccgaggagga caatgtcaag ctgctgaagt
2401 tctggatgac tggcctgagc aagacctaca agtcacacct catgtccacg gtccgcagcc
2461 ccacagcctc ggagtctcgg aactgacccg tcacacacac ctgcctaaag acagggatgg
2521 ctgtccacag gatcctccag ccccgtgaga gggactgtcc cttgagtttc tcaactgctg
2581 gaaggagctg tgtcccagca aggaagggaa accatcaggg ctgggctcgg ccctgtcagg
2641 tttggggcct gtgtgcttcc cagactctcc ctccagccgt tggaatcgct gaagatggca
2701 atgaaaggcg gagggatgat gggctctctc tgtgttcaaa ctccttggag agacgactag
2761 gaggacagct tgcctcccag gccccttgtg gacttagact caaaacccgc aggagaaaca
2821 ggtccgactc agtatgcagt cgcaataaca tgtctgctcc cgaggttaac attcaagcgt
2881 ttctactttg aaattcagca agagtttctg ggccttatgt ttgagggtac cttttgctgc
2941 agttgtgaat attcagtaca ttgccagctc ttggtcactg agtgattgag ttagggctcc
3001 gcaagagact ttggggagtg aagtggatct cttcctcatc ttttggtcct ctgaaatgtg
3061 tgttctgaag ccatggggct cgtcttctgg ggtgttcccc tgcaggtgct ggtgaaggta
3121 acctggggct taatgatgga gtccctgatc atttttgcac aagacaggtt gctgaggggt
3181 cggcaagcat ctgacttgcc caatcccctg gatatggtga gccccgccat gcttttattc
3241 tgtatcgctt ttgtctttat tgctgctttc aacatttacg tttggttaca gttaactatt
3301 ttcggagtgt ggtgattgaa gacaatttca tcatcccact gtactttttt ttttgagagg
3361 gagtttcact cttgttgccc aggctggagt gcaatggcac gatcttggct cactgcaacc
3421 tctgcctcct gggttcaagc aattctcctg cctcagcctc cagagtagct ggaactacag
3481 gtgcccgcca ctatgcccag ctaatttttg tattttttag tagagacggg gtttcaccgt
3541 gttggccggg ctggtctcaa actcctgacc tcaggtgatc cacccacctc agcctcccaa
3601 agtgctggga ttacaagcgt gagccactgt gcctggccct tttttttttt tttttttttt
3661 tttttaaaga gatggcatct tgctatgtcg tccaggctgg tcttgaactc ctgagttcaa
3721 gcagtcctcc tgcttcaaca tacagctaca ggtacccccc actatacatt tttaataagg
3781 attcatggct cagagggatt ttctgatggt tttgctgatt tgtttctagt ttttttgtgt
3841 ttatatttaa catgaagacc aagtttatat aactaggtat ctgtataatg caacaacatt
3901 ggaacacaat aaagatgtat ttttgtaaa
//
```

Nucleic Acid Sequence of FLCN transcript variant 4 (SEQ ID NO: 8):

```
LOCUS New_DNA 3950 bp ds-DNA linear
COMMENT ApEinfo:methylated:1
ORIGIN
      1 ggcggggctg cgggaccgcg agtgagtgtg gtcgctcctg gttctgccag ctcccctgag
    61 agcctgaacc cgggcttgag agcctcgcca ccccgggtga catccctgcc gtgggcttgg
  121 gggctctggg tgtgattccg ccggtccggg tcccgcagcg accacctacc cagcgcagtc
  181 aggggcgggg ctgggaccca gagcgggacc ccggctgccg agtccaggtg tcccgcgggc
  241 ctcgatttgg ggagcagaaa acgccaggtc ttcaagggtg tctgccacca ccatgcctga
  301 cccatttggc agcagcctcg tgtgtggtgg tctggtgtgg acggtggaag cgtgattctg
  361 ctgagtgtca gtgtgaccac tcgtgctcag ccgtatctca gcaggaggac aggtgccgga
  421 gcagctcgtg cagctaagca gccaactgca gaaacgtcag aacccccaaa gcctccccag
  481 tctctgataa cacatccagg gtctttcggt ggcctggagg agctataaga tctgcttcct
  541 acagcctcca ccctgtctgc tgctgctctt tggacaccgg accttacaca cccctcagac
  601 agtacagcac ccaccctgcc cttcaactct tgcttggccc cgtaagccag tccttccagg
  661 ctcctgcctc ctctggtcat tccctgcgtg ggcctctcgc aggaagcagt tttcctgtgt
  721 cccccgccga ccccctgcag gaggcctgtt gcagtctcca aggcaccatg aatgccatcg
  781 tggctctctg ccacttctgc gagctccacg gcccccgcac tctcttctgc acggaggtgc
  841 tgcacgcccc acttcctcaa ggggatggga atgaggacag tcctggccag ggtgagcagg
  901 cggaagaaga ggaaggtggc attcagatga acagtcggat gcgtgcgcac agccccgcag
  961 agggggccag cgtcgagtcc agcagcccgg ggcccaaaaa gtcggacatg tgcgagggct
1021 gccggtcact tgctgcaggg cacccgggat atatcagcca tgataaagag acctccatta
1081 aatacgtcag ccaccagcac cccagccacc cccagctctt cagcattgtc cgccaggcct
1141 gtgtccggag cctgagctgt gaggtctgcc ctggccgtga aggccccatc ttcttcggag
1201 atgagcagca cggctttgtg ttcagccaca ccttcttcat caaggacagc ctggccaggg
1261 gcttccagcg ctggtacagc atcatcacca tcatgatgga ccggatctac ctcatcaact
1321 cctggccctt cctgctgggg aaggtccggg gaatcatcga tgagctccag ggcaaggcgc
1381 tcaaggtgtt tgaggcagag cagtttggat gcccacagcg tgctcagagg atgaacacag
1441 ccttcacgcc attcctacac cagaggaacg gcaacgccgc ccgctcgctg acatcgctga
1501 caagtgatga caacctgtgg gcgtgcctgc acacctcctt tgcctggctc ctgaaggcgt
1561 gtggcagccg gctgaccgag aagctcctgg aaggtgctcc gaccgaggat accttggtcc
1621 agatggagaa gctcgctgat ttagaagagg aatcagaaag ctgggacaac tctgaggctg
1681 aagaggagga gaaagcccct gtgttgccag agagtacaga agggcgggag ctgacccagg
1741 gcccggcaga gtcctcctct ctctcaggct gtgggagctg gcagccccgg aagctgccag
1801 tcttcaagtc cctccggcac atgaggcagg tcctgggtgc cccttctttc cgcatgctgg
1861 cctggcacgt tctcatgggg aaccaggtga tctggaaaag cagagacgtg gacctcgtcc
1921 agtcagcttt tgaagtactt cggaccatgc ttcccgtggg ctgcgtccgc atcatcccat
1981 acagcagcca gtacgaggag gcctatcggt gcaacttcct ggggctcagc ccgcacgtgc
2041 agatcccccc ccacgtgctc tcctcagagt ttgctgtcat cgtggaggtc cacgcagccg
2101 cacgttccac cctccaccct gtggggtgtg aggatgacca gtctctcagc aagtacgagt
2161 ttgtggtgac cagtgggagc cctgtagctg cagaccgagt gggccccacc atcctgaata
2221 agattgaagc ggctctgacc aaccagaacc tgtctgtgga tgtggtggac cagtgcctcg
2281 tctgcctcaa ggaggagtgg atgaacaaag tgaaggtgct ttttaagttc accaaggtgg
2341 acagtcgacc caaagaggac acacagaagc tgctgagcat cctgggtgcg tccgaggagg
2401 acaatgtcaa gctgctgaag ttctggatga ctggcctgag caagacctac aagtcacacc
2461 tcatgtccac ggtccgcagc cccacagcct cggagtctcg gaactgaccc gtcacacaca
2521 cctgcctaaa gacagggatg gctgtccaca ggatcctcca gccccgtgag agggactgtc
2581 ccttgagttt ctcaactgct ggaaggagct gtgtcccagc aaggaaggga aaccatcagg
2641 gctgggctcg gccctgtcag gtttggggcc tgtgtgcttc ccagactctc cctccagccg
2701 ttggaatcgc tgaagatggc aatgaaaggc ggagggatga tgggctctct ctgtgttcaa
2761 actccttgga gagacgacta ggaggacagc ttgcctccca ggccccttgt ggacttagac
2821 tcaaaacccg caggagaaac aggtccgact cagtatgcag tcgcaataac atgtctgctc
2881 ccgaggttaa cattcaagcg tttctacttt gaaattcagc aagagtttct gggccttatg
2941 tttgagggta ccttttgctg cagttgtgaa tattcagtac attgccagct cttggtcact
3001 gagtgattga gttagggctc cgcaagagac tttggggagt gaagtggatc tcttcctcat
3061 cttttggtcc tctgaaatgt gtgttctgaa gccatggggc tcgtcttctg gggtgttccc
3121 ctgcaggtgc tggtgaaggt aacctggggc ttaatgatgg agtccctgat catttttgca
3181 caagacaggt tgctgagggg tcggcaagca tctgacttgc ccaatcccct ggatatggtg
3241 agccccgcca tgcttttatt ctgtatcgct tttgtcttta ttgctgcttt caacatttac
3301 gtttggttac agttaactat tttcggagtg tggtgattga agacaatttc atcatcccac
3361 tgtacttttt tttttgagag ggagtttcac tcttgttgcc caggctggag tgcaatggca
3421 cgatcttggc tcactgcaac ctctgcctcc tgggttcaag caattctcct gcctcagcct
3481 ccagagtagc tggaactaca ggtgcccgcc actatgccca gctaattttt gtatttttta
3541 gtagagacgg ggtttcaccg tgttggccgg gctggtctca aactcctgac ctcaggtgat
3601 ccacccacct cagcctccca aagtgctggg attacaagcg tgagccactg tgcctggccc
3661 tttttttttt tttttttttt ttttttaaag agatggcatc ttgctatgtc gtccaggctg
3721 gtcttgaact cctgagttca agcagtcctc ctgcttcaac atacagctac aggtaccccc
3781 cactatacat ttttaataag gattcatggc tcagagggat tttctgatgg ttttgctgat
3841 ttgtttctag tttttttgtg tttatattta acatgaagac caagtttata taactaggta
3901 tctgtataat gcaacaacat tggaacacaa taaagatgta tttttgtaaa
//
```

Nucleic Acid Sequence of FLCN transcript variant 5 (SEQ ID NO: 9):

```
LOCUS New_DNA 3866 bp ds-DNA linear
COMMENT ApEinfo:methylated:1
ORIGIN
      1 ggcggggctg cgggaccgcg agtgagtgtg gtcgctcctg gttctgccag ctcccctgag
    61 agcctgaacc cgggcttgag agcctcgcca ccccgggtga catccctgcc gtgggcttgg
  121 gggctctggg tgtgattccg ccggtccggg tcccgcagcg accacctacc cagcgcagtc
  181 aggggcgggg ctgggaccca gagcgggacc ccggctgccg agtccaggtg tcccgcgggc
  241 ctcgatttgg ggagcagaaa acgccaggtc ttcaagggtg tctgccacca ccatgcctga
  301 cccatttggc agcagcctcg tgtgtggtgg tctggtgtgg acggtggaag cgtgattctg
  361 ctgagtgtca gtgtgaccac tcgtgctcag ccgtatctca gcaggaggac aggtgccgga
  421 gcagctcgtg cagctaagca gccaactgca gaaacgtcag cctccaccct gtctgctgct
  481 gctctttgga caccggacct tacacacccc tcagacagta cagcacccac cctgcccttc
  541 aactcttgct tggccccgta agccagtcct tccaggctcc tgcctcctct ggtcattccc
  601 tgcgtgggcc tctcgcagga agcagttttc ctgtgtcccc cgccgacccc ctgcaggagg
  661 cctgttgcag tctccaaggc accatgaatg ccatcgtggc tctctgccac ttctgcgagc
  721 tccacggccc ccgcactctc ttctgcacgg aggtgctgca cgccccactt cctcaagggg
  781 atgggaatga ggacagtcct ggccagggtg agcaggcgga agaagaggaa ggtggcattc
  841 agatgaacag tcggatgcgt gcgcacagcc ccgcagaggg ggccagcgtc gagtccagca
  901 gcccggggcc caaaaagtcg gacatgtgcg agggctgccg gtcacttgct gcagggcacc
  961 cgggatatat cagccatgat aaagagacct ccattaaata cgtcagccac cagcacccca
1021 gccaccccca gctcttcagc attgtccgcc aggcctgtgt ccggagcctg agctgtgagg
1081 tctgccctgg ccgtgaaggc cccatcttct tcggagatga gcagcacggc tttgtgttca
1141 gccacacctt cttcatcaag gacagcctgg ccaggggctt ccagcgctgg tacagcatca
1201 tcaccatcat gatggaccgg atctacctca tcaactcctg gcccttcctg ctggggaagg
1261 tccggggaat catcgatgag ctccagggca aggcgctcaa ggtgtttgag gcagagcagt
1321 ttggatgccc acagcgtgct cagaggatga acacagcctt cacgccattc ctacaccaga
1381 ggaacggcaa cgccgcccgc tcgctgacat cgctgacaag tgatgacaac ctgtgggcgt
1441 gcctgcacac ctcctttgcc tggctcctga aggcgtgtgg cagccggctg accgagaagc
1501 tcctggaagg tgctccgacc gaggatacct tggtccagat ggagaagctc gctgatttag
1561 aagaggaatc agaaagctgg gacaactctg aggctgaaga ggaggagaaa gcccctgtgt
1621 tgccagagag tacagaaggg cgggagctga cccagggccc ggcagagtcc tcctctctct
1681 caggctgtgg gagctggcag ccccggaagc tgccagtctt caagtccctc cggcacatga
1741 ggcaggtcct gggtgcccct tctttccgca tgctggcctg gcacgttctc atggggaacc
1801 aggtgatctg gaaaagcaga gacgtggacc tcgtccagtc agcttttgaa gtacttcgga
1861 ccatgcttcc cgtgggctgc gtccgcatca tcccatacag cagccagtac gaggaggcct
1921 atcggtgcaa cttcctgggg ctcagcccgc acgtgcagat ccccccccac gtgctctcct
1981 cagagtttgc tgtcatcgtg gaggtccacg cagccgcacg ttccaccctc caccctgtgg
2041 ggtgtgagga tgaccagtct ctcagcaagt acgagtttgt ggtgaccagt gggagccctg
2101 tagctgcaga ccgagtgggc cccaccatcc tgaataagat tgaagcggct ctgaccaacc
2161 agaacctgtc tgtggatgtg gtggaccagt gcctcgtctg cctcaaggag gagtggatga
2221 acaaagtgaa ggtgcttttt aagttcacca aggtggacag tcgacccaaa gaggacacac
2281 agaagctgct gagcatcctg ggtgcgtccg aggaggacaa tgtcaagctg ctgaagttct
2341 ggatgactgg cctgagcaag acctacaagt cacacctcat gtccacggtc cgcagcccca
2401 cagcctcgga gtctcggaac tgacccgtca cacacacctg cctaaagaca gggatggctg
2461 tccacaggat cctccagccc cgtgagaggg actgtccctt gagtttctca actgctggaa
2521 ggagctgtgt cccagcaagg aagggaaacc atcagggctg ggctcggccc tgtcaggttt
2581 ggggcctgtg tgcttcccag actctccctc cagccgttgg aatcgctgaa gatggcaatg
2641 aaaggcggag ggatgatggg ctctctctgt gttcaaactc cttggagaga cgactaggag
2701 gacagcttgc ctcccaggcc ccttgtggac ttagactcaa aacccgcagg agaaacaggt
2761 ccgactcagt atgcagtcgc aataacatgt ctgctcccga ggttaacatt caagcgtttc
2821 tactttgaaa ttcagcaaga gtttctgggc cttatgtttg agggtacctt ttgctgcagt
2881 tgtgaatatt cagtacattg ccagctcttg gtcactgagt gattgagtta gggctccgca
2941 agagactttg gggagtgaag tggatctctt cctcatcttt tggtcctctg aaatgtgtgt
3001 tctgaagcca tggggctcgt cttctggggt gttcccctgc aggtgctggt gaaggtaacc
3061 tggggcttaa tgatggagtc cctgatcatt tttgcacaag acaggttgct gaggggtcgg
3121 caagcatctg acttgcccaa tcccctggat atggtgagcc ccgccatgct tttattctgt
3181 atcgcttttg tctttattgc tgctttcaac atttacgttt ggttacagtt aactattttc
3241 ggagtgtggt gattgaagac aatttcatca tcccactgta cttttttttt tgagagggag
3301 tttcactctt gttgcccagg ctggagtgca atggcacgat cttggctcac tgcaacctct
3361 gcctcctggg ttcaagcaat tctcctgcct cagcctccag agtagctgga actacaggtg
3421 cccgccacta tgcccagcta atttttgtat tttttagtag agacggggtt tcaccgtgtt
3481 ggccgggctg gtctcaaact cctgacctca ggtgatccac ccacctcagc ctcccaaagt
3541 gctgggatta caagcgtgag ccactgtgcc tggccctttt tttttttttt tttttttttt
3601 ttaaagagat ggcatcttgc tatgtcgtcc aggctggtct tgaactcctg agttcaagca
3661 gtcctcctgc ttcaacatac agctacaggt accccccact atacattttt aataaggatt
3721 catggctcag agggattttc tgatggtttt gctgatttgt ttctagtttt tttgtgttta
3781 tatttaacat gaagaccaag tttatataac taggtatctg tataatgcaa caacattgga
3841 acacaataaa gatgtatttt tgtaaa
//
```

What is claimed:

1. A composition for preventing or treating nonalcoholic fatty liver disease or nonalcoholic steatohepatitis in a subject comprising an inhibitor of folliculin.

2. The composition according to claim 1, wherein the inhibitor of folliculin is targeted to hepatocytes of the subject.

3. The composition according to claim 1, wherein the inhibitor of folliculin operates by RNA interference or an RNA-guide nuclease.

4. The composition according to claim 1, wherein the inhibitor of folliculin is small interfering RNA (siRNA), wherein the siRNA inhibits FLCN expression, and wherein:
the siRNA is complementary to a portion of the nucleic acid sequence of FLCN expression;
siRNA is complementary to a portion of the nucleic acid sequence of FLCN transcript variant 1-5 (SEQ ID NO: 5-9); or
the siRNA is complementary to a portion of the nucleic acid sequence encoding the amino acid sequence of SEQ ID NO: 1.

5. The composition according to claim 4, wherein the siRNA is a GalNac-modified oligonucleotide.

6. The composition according to claim 1, wherein the inhibitor of folliculin is a CRISPR-Cas9 combination comprising a Cas9 enzyme and a guide RNA (gRNA) that specifically binds to, recognizes, or hybridizes to FLCN.

7. The composition according to claim 6, wherein the guide RNA:
specifically binds to, recognizes, or hybridizes to a part of the nucleic acid sequence of FLCN transcript variant 1-5 (SEQ ID NO: 5-9);
comprises a nucleic acid that is at least 90% identical to SEQ ID NO: 2-4; or
comprises a nucleic acid of SEQ ID NO: 2-4.

8. A method for preventing or treating nonalcoholic fatty liver disease or nonalcoholic steatohepatitis in a subject comprising administering to the liver of the subject an inhibitor of folliculin.

9. The method according to claim 8, wherein the inhibitor of folliculin is targeted to hepatocytes of the subject.

10. The method according to claim 8, wherein the inhibitor of folliculin is a small interfering RNA (siRNA), wherein the siRNA inhibits FLCN expression, and wherein:
the siRNA is complementary to a portion of the nucleic acid sequence of FLCN expression;
the siRNA is complementary to a portion of the nucleic acid sequence of FLCN transcript variant 1-5 (SEQ ID NO: 5-9); or
the siRNA is complementary to a portion of the nucleic acid sequence encoding the amino acid sequence of SEQ ID NO: 1.

11. The method according to claim 10, wherein the siRNA is a GalNac-modified oligonucleotide.

12. The method according to claim 8, wherein the inhibitor of folliculin is a CRISPR-Cas9 combination comprising a Cas9 enzyme and a guide RNA (gRNA) that specifically binds to, recognizes, or hybridizes to a part of FLCN.

13. The method according to claim 12, wherein the guide RNA:
specifically binds to, recognizes, or hybridizes to a part of the nucleic acid sequence of FLCN transcript variant 1-5 (SEQ ID NO: 5-9);
comprises a nucleic acid that is at least 90% identical to SEQ ID NO: 2-4; or
comprises a nucleic acid of SEQ ID NO: 2-4.

14. A method for preventing or treating nonalcoholic fatty liver disease or nonalcoholic steatohepatitis in a subject comprising silencing or down-regulating FLCN in hepatocytes of the subject.

15. The method according to claim 14, wherein FLCN is silenced or down-regulated in hepatocytes of the subject using RNA interference or an RNA-guided nuclease.

16. The method according to claim 14, wherein FLCN is silenced or downregulated using a small interfering RNA (siRNA), wherein the siRNA silences or downregulates FLCN expression, and wherein:
the siRNA is complementary to a portion of the nucleic acid sequence of FLCN expression;
the siRNA is complementary to a portion of the nucleic acid sequence of FLCN transcript variant 1-5 (SEQ ID NO: 5-9); or
the siRNA is complementary to a portion of the nucleic acid sequence encoding the amino acid sequence of SEQ ID NO: 1.

17. The method according to claim 14, wherein the siRNA is a GalNac-modified oligonucleotide.

18. The method according to claim 11, wherein FLCN is silenced or downregulated using a CRISPR-Cas9 combination comprising a Cas9 enzyme and a guide RNA (gRNA) that specifically binds to, recognizes, or hybridizes to a part of FLCN.

19. The method according to claim 15, wherein the guide RNA specifically binds to, recognizes, or hybridizes to a part of the nucleic acid sequence of FLCN transcript variant 1-5 (SEQ ID NO: 5-9).

20. The method according to claim 18, wherein the guide RNA comprises a nucleic acid that is at least 90% identical to SEQ ID NO: 2-4, or a nucleic acid of SEQ ID NO: 2-4.
